(19) United States
(12) Patent Application Publication
(10) Pub. No.: US 2022/0249150 A1
(43) Pub. Date: Aug. 11, 2022

(54) DEVICES, SYSTEMS AND METHODS FOR MEASURING SKIN ELASTICITY AND PERFORMING SUBDERMAL COAGULATION TO INCREASE TISSUE FIRMNESS

(71) Applicant: Apyx Medical Corporation, Clearwater, FL (US)

(72) Inventors: Shawn D. Roman, Safety Harbor, FL (US); Amin Elachchabi, Hollywood, FL (US); Fredrik Jonsson, St. Petersburg, FL (US); Gregory Goliszek, Oldsmar, FL (US)

(21) Appl. No.: 17/616,289

(22) PCT Filed: Jun. 8, 2020
(86) PCT No.: PCT/US2020/036593

§ 371 Date: Dec. 3, 2021

Related U.S. Application Data
(60) Provisional application No. 62/859,151, filed on Jun. 9, 2019.

(51) Int. Cl. A61B 18/04; A61N 1/44; A61M 1/00

(52) U.S. Cl. CPC A61B 18/042; A61B 2018/00464; A61M 1/89; A61N 1/44

(57) ABSTRACT

The present disclosure relates to devices, systems and methods for subdermal tissue tightening through soft tissue coagulation and for use in cosmetic surgery applications. The devices, systems and methods of the present disclosure may be used for a minimally invasive application of plasma energy to subcutaneous tissue for the purpose of tightening lax tissue. The present disclosure further provides several elasticity measurement devices, which are used to determine the elasticity of a tissue. The measurements obtained by the elasticity measurement devices are used to determine when a desired firmness or elasticity has been achieved during a tissue tightening procedure.

### PRIORITY

This application claims priority to U.S. Provisional Patent Application Ser. No. 62/859,151, filed Jun. 9, 2019, entitled “DEVICES, SYSTEMS AND METHODS FOR MEASURING SKIN ELASTICITY AND PERFORMING SUBDERMAL COAGULATION TO INCREASE TISSUE FIRMNESS”, the contents of which are hereby incorporated by reference in its entirety.

### BACKGROUND

### Field

The present disclosure relates generally to electrosurgery and electrosurgical systems and apparatuses, and more particularly, devices, systems and methods for measuring skin elasticity and performing tissue tightening through soft tissue coagulation.

### Description of the Related Art

High frequency electrical energy has been widely used in surgery and is commonly referred to as electrosurgical energy. Tissue is cut and bodily fluids are coagulated using electrosurgical energy.

Gas plasma is an ionized gas capable of conducting electrical energy. Plasmas are used in surgical devices to conduct electrosurgical energy to a patient. The plasma conducts the energy by providing a pathway of relatively low electrical resistance. The electrosurgical energy will follow through the plasma to cut, coagulate, desiccate, or fulgurate blood or tissue of the patient. There is no physical contact required between an electrode and the tissue treated.

Electrosurgical systems that do not incorporate a source of regulated gas can ionize the ambient air between the active electrode and the patient. The plasma that is thereby created will conduct the electrosurgical energy to the patient, although the plasma arc will typically appear more spatially dispersed compared with systems that have a regulated flow of ionizable gas.

Atmospheric pressure discharge cold plasma applicators have found use in a variety of applications including surface sterilization, hemostasis, and ablation of tumors. Often, a simple surgical knife is used to excise the tissue in question, followed by the use of a cold plasma applicator for cauterization, sterilization, and hemostasis. Cold plasma beam applicators have been developed for both open and endoscopic procedures. In the latter case, it is often desirable to be able to redirect the position of the cold plasma beam tip to a specific operative site. The external incision and pathway for the endoscopic tool may be chosen to avoid major blood vessels and non-target organs and may not coincide with an optimum alignment for the target internal tissue site. A means of redirecting the cold plasma beam is essential in these situations.

The heat effects of the radiofrequency (RF) alternating current used in electrosurgery on cells and tissue have been well established. Normal body temperature is 37° C. and, with normal illness, can increase to 40° C. without permanent impact or damage to the cells of our body. However, when the temperature of cells in tissue reaches 50° C., cell death occurs in approximately 6 minutes. When the temperature of cells in tissue reaches 60° C., cell death occurs instantaneously. Between the temperatures of 60° C. and just below 100° C., two simultaneous processes occur. The first is protein denaturation leading to coagulation which will be discussed in more detail below. The second is desiccation or dehydration as the cells lose water through the thermally damaged cellular wall. As temperatures rise above 100° C., intracellular water turns to steam and tissue cells begin to vaporize as a result of the massive intracellular expansion that occurs. Finally, at temperatures of 200° C. or more, organic molecules are broken down into a process called carbonization. This leaves behind carbon molecules that give a black and/or brown appearance to the tissue.

Understanding these heat effects of RF energy on cells and tissue can allow the predictable changes to be used to accomplish beneficial therapeutic results. Protein denaturation leading to soft tissue coagulation is one of the most versatile and widely utilized tissue effects. Protein denaturation is the process in which hydrothermal bonds (i.e., crosslinks) between protein molecules, such as collagen, are instantaneously broken and then quickly reformed as tissue cools. This process leads to the formation of uniform clumps of protein typically called coagulum through a subsequent process known as coagulation. In the process of coagulation, cellular proteins are altered but not destroyed and form protein bonds that create homogenous, gelatinous structures. The resulting tissue effect of coagulation is extremely useful and most commonly used for occluding blood vessels and causing hemostasis.

In addition to causing hemostasis, coagulation results in contraction of soft tissue. Collagen is one of the main proteins found in human skin and connective tissue. The coagulation/denaturation temperature of collagen is conventionally stated to be 66.8° C., although this can vary for different tissue types. Once denatured, collagen rapidly contracts as fibers shrink to one-third of their overall length. However, the amount of contraction is dependent upon the temperature and the duration of the treatment. The hotter the temperature the shorter amount of treatment time needed for maximal contraction. For example, collagen heated at a temperature of 65° C. must be heated for greater than 120 seconds for significant contraction to occur.

Thermal-induced contraction of collagen through the coagulation of soft tissue is used in ophthalmology, orthopedic applications, and the treatment of varicose veins. The reported range of temperatures causing collagen contraction varies from 60° C. to 80° C. Therefore, once tissue is heated to within this range, protein denaturation and collagen contraction occur resulting in the reduction in volume and surface area of the heated tissue. Noninvasive radiofrequency devices, lasers, and plasma devices have been used for the reduction of facial wrinkles and rhytides caused by thermal-induced collagen/tissue contraction.

Although tissue heating devices may be effective in achieving soft tissue contraction, the evidence has been primarily limited to volume or surface area changes as measured through photographic analysis of before and after images. Measurement of the changes in biomechanical properties of the tissue resulting from soft tissue coagulation and contraction has proven more difficult. Therefore, a need exists for devices, systems, and methods capable of producing accurate measurements of biomechanical properties of tissue.

### SUMMARY

The present disclosure relates to devices, systems and methods for tissue tightening, both the skin surface and subdermal tissue planes of interest, through soft tissue coagulation for use in cosmetic surgery applications. The devices, systems and methods of the present disclosure may be used for a minimally invasive application of plasma energy to subcutaneous tissue for the purpose of tightening lax skin surface tissue. The present disclosure provides several skin elasticity measurement devices, which are used to determine the elasticity of a skin surface. The measurements obtained by the skin elasticity measurement devices are used to determine when a desired skin firmness has been achieved during a skin tightening procedure.

In one aspect of the present disclosure, a method is provided including performing an elasticity altering procedure on a subcutaneous tissue plane; measuring elasticity of the altered tissue; determining if the elasticity measurement is within a threshold value; and iteratively performing an elasticity altering procedure on the subcutaneous tissue plane until the elasticity measurement is within the threshold.

In another aspect, the method further includes determining the threshold value by measuring elasticity of the portion of the tissue before performing the elasticity altering procedure.

In one aspect, the elasticity altering procedure is a liposuction procedure.

In a further aspect, the elasticity altering procedure involves applying energy to heat the tissue plane.

In another aspect, the applying energy to heat the tissue plane includes applying a plasma beam to the tissue plane.

In yet another aspect, the tissue plane is heated to a temperature of at least 85 degrees C. for about 0.04 seconds to about 0.08 seconds.

In one aspect, a power output of the plasma beam is about 24 Watts to about 32 Watts.

In a further aspect, the measuring elasticity of the portion of the tissue plane includes: measuring stress at a plurality of displacements of the tissue to obtain a plurality of data points; and determining the slope of the data points.

In another aspect, the determining if the elasticity measurement is within a threshold value includes comparing the determined slope to the threshold value.

In a further aspect, an initial elasticity altering procedure is a liposuction procedure and subsequent elasticity altering procedures include applying a plasma beam to the tissue plane.

In one aspect of the present disclosure, a system is provided including an applicator for applying a plasma beam to a tissue plane, the tissue plane beneath a portion of a surface of skin and receiving an elasticity altering procedure; an elasticity measurement device for measuring elasticity of the portion of tissue; and an electrosurgical generator unit for supplying electrosurgical energy to the applicator, determining if the elasticity measurement is within a threshold value and iteratively applying the plasma beam to the tissue plane until the elasticity measurement is within the threshold.

In another aspect, the threshold value is the elasticity measurement of the portion of the tissue before the elasticity altering procedure is performed.

In a further aspect, the elasticity measurement device measures elasticity by measuring stress at a plurality of displacements of the tissue to obtain a plurality of data points and determining the slope of the data points.

In one aspect, the electrosurgical generator unit determines if the elasticity measurement is within the threshold value by comparing the determined slope to the threshold value.

In yet another aspect, the elasticity measurement device is at least one of a tensile testing device, a vacuum assisted device, a lateral shear measurement device and/or a lateral load measurement device.

In one aspect, the elasticity measurement device measures back pressure generated by gas introduced percutaneously to the tissue plane.

In a further aspect, the elasticity measurement device measures changes in density of a contrast fluid introduced into the tissue plane.

In one aspect, the elasticity measurement device is pressure sensitive forceps.

In another aspect, the elasticity measurement device is ultrasound device that introduces sound waves into the tissue plane and measures returning sounds waves propagating through the tissue plane.

In still another aspect, the elasticity measurement device includes at least one inflatable element, the at least one inflatable element includes a pressure sensor.

In another aspect of the present disclosure, an elasticity measurement device includes a shaft having a first end and a second end, a gauge coupler disposed at the first end and a gripping member disposed in and extending from the second end, the gripping member coupled within the shaft to the gauge coupler, a base coupled to the second end of the shaft, the base including a ring disposed a predetermined distance from the second end of the shaft, wherein the ring is disposed over a portion of tissue enabling the gripping member to be coupled to the tissue and the tissue drawn into an aperture formed by the ring; and a handle coupled to the first end of the shaft and configured for receiving a force gauge, the force gauge being coupled to the gauge coupler such that when a force is applied to the gripping member in a direction opposite the first end, the force is transferred to gauge coupler and measured by the force gauge, wherein the force gauge measures the force at various displacements of the gripping member.

In another aspect, the device further includes a motor coupled to the shaft to extend and retract the second end of the shaft relative to the base.

In one aspect, the device further includes an electromagnetic mechanism coupled to the shaft to extend and retract the second end of the shaft relative to the base.

In another aspect, the gripping member is configured to grip the skin surface directly.

In a further aspect, the gripping member is configured to be attached to an interface that is attached to the tissue surface.

In yet another aspect, the interface is at least one of suture loops placed through the tissue and/or a pad having an adhesive backing.

In another aspect, the shaft is configured a first tubular member and a second tubular member, a first end of first tubular member is coupled to the handle and a second end of first tubular member is disposed through a first end of second tubular member, such that the first and second tubular members are slidable with respect to each other and a second end of the second tubular member is coupled to the base, wherein the first or second tubular members include a plurality of markings to indicate the position and displacement of the first tubular member relative to the second tubular member.

In a further aspect of the present disclosure, a device is provided including a housing that receives electrosurgical energy for supplying the energy to an electrode, the electrode being disposed in a shaft; the shaft including a proximal end and a distal end, the proximal end of the shaft being pivotly coupled to the housing, wherein plasma is applied to a tissue plane of interest via the distal end of the shaft; and a strain sensor configured to sense strain imposed on shaft when pivoted relative to the housing.

In one aspect, the device further includes a load cell disposed on the distal end of the shaft and configured to measure a force applied to the distal end of the shaft when in contact with tissue.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1A is an illustration of an exemplary electrosurgical system in accordance with an embodiment of the present disclosure;

FIG. 1B is an illustration of an exemplary electrosurgical generator unit in accordance with an embodiment of the present disclosure;

FIG. 1C is a flowchart illustrating an exemplary method for tightening tissue in accordance with an embodiment of the present disclosure;

FIG. 1D is a graph including stress-strain curves for human skin in accordance with an embodiment of the present disclosure;

FIG. 1E is a flowchart illustrating an exemplary method for tightening tissue and measuring skin elasticity in accordance with an embodiment of the present disclosure;

FIG. 1F is a graph illustrating mean tissue firmness per stage of tissue tightening treatment in accordance with an embodiment of the present disclosure;

FIGS. 2A-2D include various views of a skin elasticity measurement device including a motor for pulling skin in a vertical direction in accordance with an embodiment of the present disclosure;

FIG. 3 is a perspective view of a skin elasticity measurement device for manually pulling skin in a vertical direction in accordance with an embodiment of the present disclosure;

FIG. 4 is a perspective view of a vacuum assisted skin elasticity measurement device in accordance with an embodiment of the present disclosure;

FIG. 5 is a perspective view of a skin elasticity measurement device including at least one adhesive in accordance with an embodiment of the present disclosure;

FIGS. 6A-6D include various views of another skin elasticity measurement device including at least one adhesive in accordance with an embodiment of the present disclosure;

FIG. 7A illustrates a system for measuring skin elasticity using pulses of inert gas introduced percutaneously into a tissue plane of interest in accordance with an embodiment of the present disclosure;

FIG. 7B illustrates a ring for use with the system of FIG. 7A in accordance with an embodiment of the present disclosure;

FIGS. 8A, 8B illustrate a system for measuring skin elasticity using contrast fluid injected into a tissue pane of interest in accordance with an embodiment of the present disclosure;

FIG. 9 illustrates a skin elasticity measurement device configured as pressure sensitive forceps in accordance with an embodiment of the present disclosure;

FIGS. 10A-10C illustrate a skin elasticity measurement device configured as a clamp or forceps including pressure sensitive tips in accordance with an embodiment of the present disclosure;

FIGS. 11A-11C illustrate a skin elasticity measurement device configured in a T-rod shape in accordance with an embodiment of the present disclosure;

FIGS. 12A, 12B illustrate another skin elasticity measurement device configured as pressure sensitive forceps in accordance with an embodiment of the present disclosure;

FIG. 13A illustrates a skin elasticity measurement device including ultrasound detection capabilities in accordance with an embodiment of the present disclosure;

FIG. 13B illustrates a plasma applicator or handpiece including an ultrasound transducer in accordance with an embodiment of the present disclosure;

FIG. 13C illustrates a plasma applicator or handpiece including a strain sensor for measuring strain on a shaft of the plasma applicator in accordance with an embodiment of the present disclosure;

FIG. 13D illustrates a plasma applicator or handpiece including a strain sensor and an ultrasound transducer in accordance with an embodiment of the present disclosure;

FIG. 14 illustrates a skin elasticity measurement device configured to use the effect of gravity on tissue of interest to measure skin elasticity in accordance with an embodiment of the present disclosure;

FIGS. 15A, 15B illustrate a skin elasticity measurement device configured to be inflated in a subcutaneous tissue plane in accordance with an embodiment of the present disclosure; and

FIG. 16 illustrates a skin elasticity measurement device configured to measure the load of a portion of a patient's anatomy before, during, and after a skin elasticity altering procedure in accordance with an embodiment of the present disclosure.

It should be understood that the drawings are for purposes of illustrating the concepts of the disclosure and are not necessarily the only possible configuration for illustrating the disclosure.

### DETAILED DESCRIPTION

Preferred embodiments of the present disclosure will be described hereinbelow with reference to the accompanying drawings. In the following description, well-known functions or constructions are not described in detail to avoid obscuring the present disclosure in unnecessary detail. In the drawings and in the description which follow, the term “proximal”, as is traditional, will refer to the end of the device, e.g., instrument, apparatus, applicator, handpiece, forceps, etc., which is closer to the user, while the term “distal” will refer to the end of the device which is further from the user. Herein, the phrase “coupled” is defined to mean directly connected to or indirectly connected with through one or more intermediate components. Such intermediate components may include both hardware and software based components.

The use of thermal-induced collagen/tissue contraction has been expanded to minimally invasive procedures. Laser-assisted lipolysis (LAL) and radiofrequency-assisted lipolysis (RFAL) devices have combined the removal of subcutaneous fat with soft tissue heating to reduce the skin laxity that often results from fat volume removal. These devices are placed in the same subcutaneous tissue plane as a standard suction-assisted lipolysis (SAL) cannula and are used to deliver thermal energy to coagulate the subcutaneous tissue including the underside of the dermis, the fascia, and the septal connective tissue. The coagulation of the subcutaneous tissue results in collagen/tissue contraction that reduces skin laxity.

The devices, systems and methods of the present disclosure are employed for the minimally invasive application of helium-based (or based on other inert gases) cold plasma energy to subcutaneous tissue for the purpose of tightening lax tissue. A tip of a plasma generating handpiece is placed in the subcutaneous tissue plane through the same access ports used for SAL. Activation of the plasma generating handpiece in this plane causes contraction of the collagen contained in the dermis, the fascia, and the septal connective matrix through precise heating from the plasma energy.

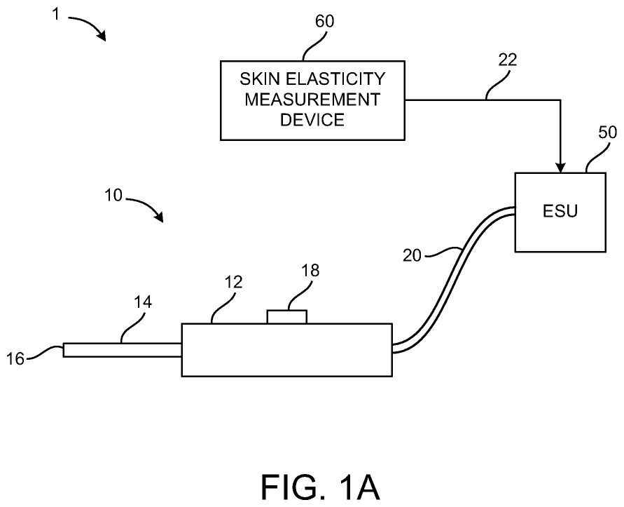

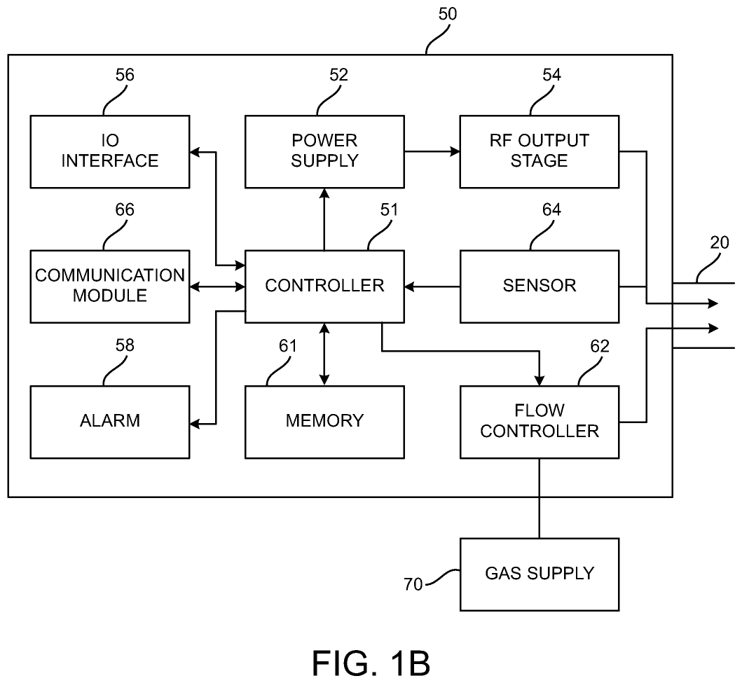

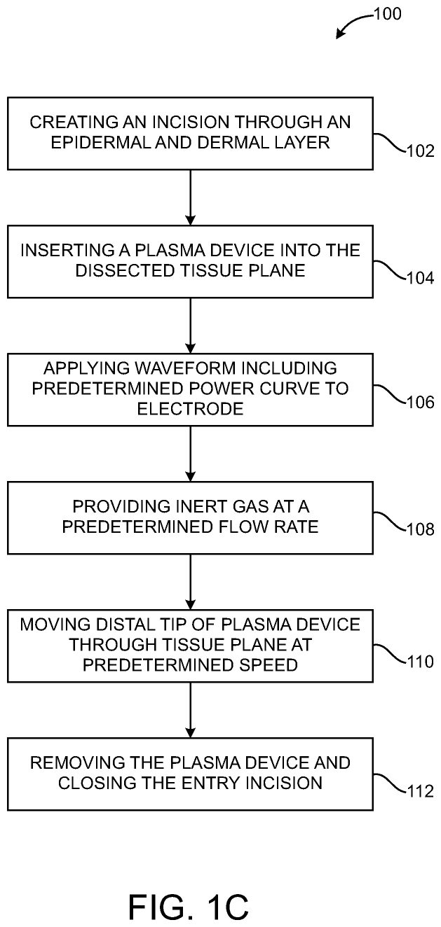

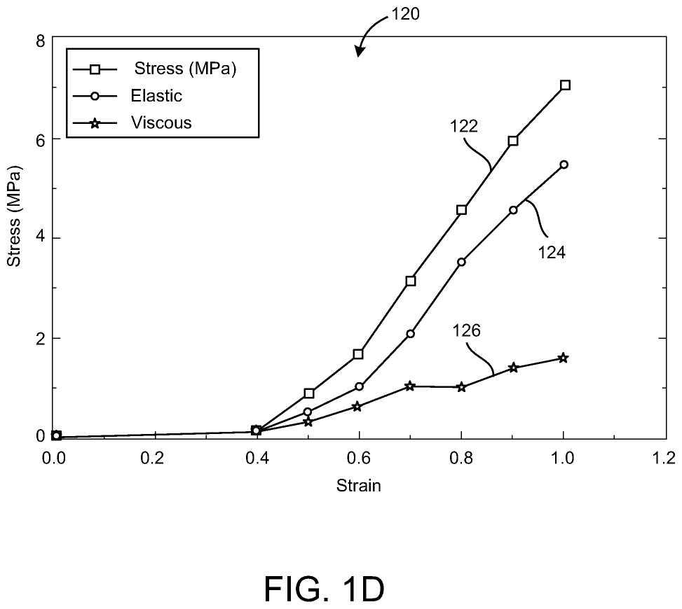

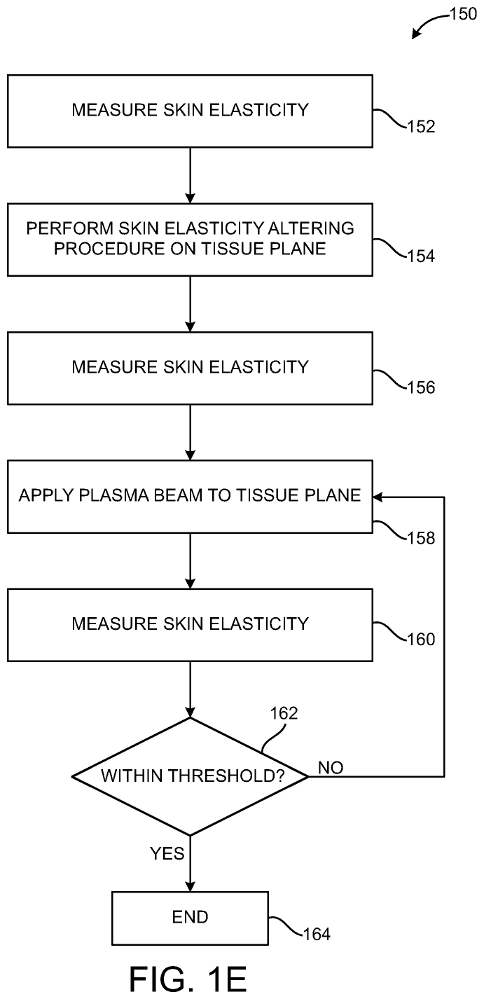

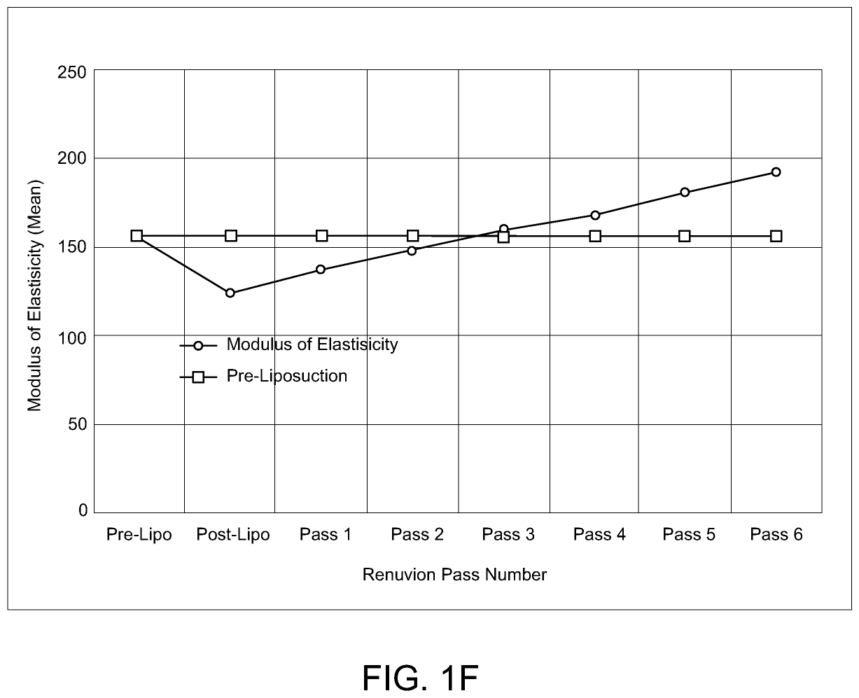

For example, referring to FIG. 1A, an electrosurgical system 1 is shown in accordance with the present disclosure. System 1 includes a plasma applicator or handpiece 10, an electrosurgical generator unit (ESU) 50, and a skin elasticity measurement device 60.

Applicator 10 is configured to receive electrosurgical energy from ESU 50 via a cable 20. Applicator 10 is further configured to receive an inert gas from a gas source. In some embodiments, the inert gas is provided from ESU 50 via cable 20. In other embodiments, the inert gas is provided via a source (not shown) separately coupled to applicator 10. Applicator 10 includes a handle housing 12 having a button 18 and a shaft 14 having a distal tip 16. When button 18 is pressed electrosurgical energy is delivered to applicator 10 by ESU 50 and inert gas is delivered to applicator 10 by the gas source. The electrosurgical energy is used to energize an electrode disposed in shaft 14, e.g., a wire electrode, a blade electrode, a needle electrode, etc. When the inert gas, is passed over the energized electrode, a plasma is generated and emitted from tip 16 to patient tissue, which allows for conduction of the RF energy from the electrode to the patient in the form of a precise plasma beam. An exemplary applicator for generating plasma is shown and described in commonly owned U.S. Pat. No. 9,060,765, the contents of which are incorporated by reference. In one embodiment, Helium is used as the inert gas because it can be converted to a plasma with very little energy. With the devices and systems of the present disclosure, less than 0.1% of the Helium gas employed is converted to plasma, so >99.9% of the Helium remains in a gaseous state. In some embodiments, the skin elasticity measurement device 60 may be coupled to electrosurgical generator 50 via a communication medium, via hardwired or wireless.

Referring to FIG. 1B, a block diagram of an exemplary ESU 50 is shown in accordance with an embodiment of the present disclosure. ESU 50 may include a controller or processor 51, power supply 52, radio frequency (RF) output stage 54, I/O interface 56, alarm 58, memory 61, flow controller 62, sensor 64, and a communication module 66. Controller 51 is configured to control power supply 52 to supply electrosurgical energy being output from RF output stage 54 via at least one conductor extending through cable 20 to the applicator 10. I/O interface 56 is configured to receive user input (e.g., via one or more buttons, touchscreens, etc., disposed on the housing of ESU 50) and output information (e.g., data, graphical user interfaces, etc.) received from controller 51. Audible alarm 58 is controllable via controller 51 to alert an operator to various conditions or events. Flow controller 62 is configured for controlling the flow of gas received from supply 70 to the applicator 10. The flow controller 62 is coupled to the controller 51 and receives control signals from the controller 51 based on user input via I/O interface 56 or based on an algorithm or software function stored in memory 61. Although in the embodiment shown in FIG. 1B, the flow controller 62 is disposed in the ESU 50, the flow controller 62 can be located external to the ESU 50 and disposed, for example, in a separate housing, in the applicator 10, etc.

Communication module 66 of ESU 50 is configured to communicate with other devices (e.g., skin elasticity measurement devices, client devices, servers, etc.) via a communication link (e.g., wired or wireless) to send and receive data and communications. In one embodiment, the communication module 66 may include a communication bus for receiving data from the skin elasticity measurement device 60 via communication medium 22, e.g., RS-485, USB, etc. In other embodiments, the communication module 60 may include a transceiver for wirelessly communicating with the skin elasticity measuring device 60. Although in the embodiment shown in FIG. 1B, an operator is alerted to various conditions via an audible alarm 58, in other embodiments, controller 51 may use communication module 66 to send notifications to at least one other device via the communication link (e.g., wired or wireless), where the communications are associated with the various conditions or events.

In one embodiment, sensor 64 of ESU 50 is coupled to the output of RF output stage 54. Sensor 64 is configured to sample the voltage and/or current (or any other electrical properties) of the output of stage 54 and provide the sample voltage and/or current to controller 51. Controller 51 may use the information to determine one or more properties associated with the power provided by ESU 50 to applicator 10.

It is to be appreciated that the functions of the ESU 50 may be provided through the use of dedicated hardware as well as hardware capable of executing software in association with appropriate software. In one embodiment, some or all of the functions may be performed by at least one processor 51, such as a computer or an electronic data processor, digital signal processor or embedded micro-controller, in accordance with code, such as computer program code, software, and/or integrated circuits that are coded to perform such functions, unless indicated otherwise. When provided by a processor 51, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, read only memory (ROM) for storing software, random access memory (RAM), and nonvolatile storage.

The unique heating of the devices and systems of the present disclosure makes it a useful surgical tool for the coagulation of subcutaneous soft tissue similar to the LAL and RFAL devices discussed above. As the tip 16 of the applicator 10 is drawn through the subdermal plane, heating of the tissue results in instant coagulation and contraction of the tissue followed by immediate cooling.

Some devices commercially available for subcutaneous soft tissue coagulation work on the principle of bulk tissue heating. In these devices, the energy is primarily directed into the dermis and the device is activated until a pre-set subdermal temperature in the range of 65° C. is achieved and maintained across the entire volume of tissue. As discussed above, at 65° C., the tissue being treated must be maintained at that temperature for greater than 120 seconds for maximal contraction to occur. Although these devices may be effective in achieving soft tissue contraction, the process of heating all of the tissue to the treatment temperature and maintaining that temperature for extended periods can be time consuming. In addition, during this process, the heat eventually conducts to the epidermis requiring constant monitoring of epidermal temperatures to ensure they do not exceed safe levels.

In contrast to previous approaches, handpiece 10 and ESU 50 of system 1 of the present disclosure achieve soft tissue coagulation and contraction by rapidly heating the treatment site to temperatures greater than 85° C. for between about 0.040 and about 0.080 seconds. It is to be appreciated that handpiece 10 and/or ESU 50 may include a processor configured to ensure the heat (provided via the tip of the applicator, e.g., tip 16) applied to patient is maintained for between about 0.040 and about 0.080 seconds. For example, when button 18 of applicator 10 is pressed, a processor in applicator 10 or processor 51 in ESU 50 may be configured to apply electrosurgical energy to the electrode continuously for between about 0.040 and about 0.080 seconds.

In some embodiments, a temperature sensor (e.g., an optical sensor, thermocouple, resistance temperature detector (RTD)) may be included in the distal tip (e.g., 16) or be otherwise in communication with the applicator 10 and/or the ESU 50. The temperature sensor provides temperature readings of the target tissue to the processor. The processor 51 is configured to adjust the power outputted by ESU 50 and the time duration that the heat is applied to the target tissue to ensure that temperatures greater than 85° C. for between about 0.040 and about 0.080 seconds are reached.

In some embodiments, a predetermined power curve is stored in memory 61 and applied by ESU 50 to the electrode in applicator 10. The predetermined power curve ensures the tissue is heated to temperatures at least 85° C. for between about 0.040 to about 0.080 seconds. Furthermore, in accordance with the present disclosure, other properties associated with the application of plasma may be controlled to guarantee the temperatures of the tissue heated. For example, the flow rate of the inert gas provided to distal tip 16 and the speed that the tip 16 is moved through the tissue plane may be selected to ensure the target temperatures described above are reached.

Referring to FIG. 1C, a method 100 of coagulating a subcutaneous layer of tissue using the handpiece or applicator 10 and ESU 50 of system 1 is shown in accordance with an embodiment of the present disclosure.

Initially, in step 102, an incision, i.e., an entry incision, is created through the epidermal and dermal layers of a patient at a location appropriate for a particular procedure. In step 104, the tip 16 of the applicator 10 is inserted into the dissected tissue plane. Next, the applicator is activated (e.g., is activated to coagulate and/or ablate tissue to create a desired effect, e.g., (i) tighten tissue (ii) shrink tissue and/or (iii) contour or sculpt the body).

When the applicator 10 is activated, in step 106, ESU 50 applies a waveform including a predetermined power curve to the electrode of applicator 10. In one embodiment, the predetermined power curve is configured such that electrosurgical energy is provided in a pulsed manner, with each pulse having a predetermined time duration and with the electrosurgical generator outputting a predetermined output power when the waveform is applied. For example, in one embodiment, the power curve is configured such that the predetermined time duration of a pulse is between about 0.04 seconds and about 0.08 seconds and the predetermined output power is between about 24 Watts and about 32 Watts.

Furthermore, when applicator 10 is activated, in step 106, a gas source (e.g., integrated with ESU 50 or separate from ESU 50) is configured to provide inert gas to the distal tip 16 of applicator 10 at a predetermined flow rate, in step 108. In one embodiment, the inert gas used is helium and the predetermined flow rate is between 1 liter per minute and 5 liters per minute.

In step 110, the user moves the distal tip 16 of the applicator 10 through the tissue plane at a predetermined speed. In one embodiment, the predetermined speed is about 1 centimeter per second. It is to be appreciated that, in the method 100, the predetermined power curve of the waveform, the predetermined flow rate of the inert gas, and the predetermined speed of the tip 16 through the tissue plane are selected such that, when steps 106-110 are performed, the temperature of the tissue being heated by the plasma emitted from the applicator 10 reaches at least 85° C. and the tissue is not heated in bulk (e.g., in areas surrounding or further away from the target tissue), but instead is heated instantaneously and cools quickly after treatment. After the desired effects are achieved, the applicator 10 is removed, and the entry incision is closed, in step 112.

Unlike with bulk tissue heating, the rapid heating of tissue performed by the system 1 of the present disclosure allows the tissue surrounding the treatment site to remain at much cooler temperatures resulting in rapid cooling after the application of the energy through conductive heat transfer. Additionally, the energy provided to the tissue using the electrosurgical apparatuses of the present disclosure is focused on heating the fibroseptal network (FSN) instead of the dermis. The majority of soft tissue contraction induced by the subcutaneous energy delivery devices is due to its effect on the FSN. Because of these unique heating and cooling properties of the electrosurgical apparatuses of the present disclosure, immediate soft tissue contraction can be achieved without unnecessarily heating the full thickness of the dermis.

Method 100 may be used to perform collagen/tissue contraction in minimally invasive, percutaneous skin tightening procedures to reduce the skin laxity or elasticity that often results from fat volume removal, e.g., during liposuction. Applicator 10 may be placed in the same subcutaneous tissue plane (or any tissue plane of interest) as a liposuction cannula and used to deliver thermal energy to coagulate the subcutaneous tissue including the septal connective tissue and, to a lesser extent, the underside of the dermis and fascia. The coagulation of the subcutaneous tissue results in collagen/tissue contraction that reduces skin laxity or elasticity.

Elasticity is defined as the ability of an object or material to resume its normal shape after being stretched or compressed. The rubber in a balloon is an example of a material that is near 100% elastic. When a balloon that was just inflated is compressed again, it will quickly spring back to its original shape. In contrast, modeling clay is a 100% plastic material. When mechanical force is applied, it will retain the resulting shape even after the force is removed. Human skin is not completely elastic or completely plastic. Skin deforms with applied force but returns to its original position after a slight delay when the force is removed. This combined elastic and plastic behavior of human tissue is called viscoelasticity. The elastic and plastic behavior of skin, connective tissue, and other materials is most commonly graphically displayed on a stress-strain curve. The stress-strain curve is unique for a given material and is established by recording the amount of deformation or displacement of the material (i.e., strain) corresponding to a tensile or compressive load (i.e., stress).

For example, referring to FIG. 1D, a graph 120 is shown including the total (curve 122), elastic (curve 124), and viscous (curve 126) stress-strain curves established for human skin.

As can be seen on the total stress-strain curve 122 in FIG. 1D, the stress-strain behavior of skin is composed of three distinct phases. In the first phase corresponding to small deformations or displacements of the tissue (i.e., strain less than or equal to 0.4), the collagen fibers and fibrils in the tissue offer little resistance to deformation as they begin to uncrimp or unfold. In the second phase corresponding to higher deformations (i.e., strains between 0.4 and 0.6), the collagen fibers begin to offer resistance to the deformation and begin to carry the load of the force applied. The stress-strain relationship during this second phase is highly linear and is dominated by the elastic characteristics of the tissue and the collagen. In the third phase corresponding to large deformations (i.e., strains greater than 0.6), the crosslinks between the collagen fibrils begin to yield and eventually failure of the tissue occurs (such as tearing).

Analyzing the stress-strain curve of skin and connective tissue during the highly linear second phase described above can provide significant insight into its elastic characteristics. For example, calculating the slope of this linear portion of the stress-strain curve provides a measure of the tissue's resistance to deformation by a mechanical force (i.e., slope=change in stress/change in strain). In material science and engineering disciplines, this resistance is most commonly referred to as the elastic modulus or the modulus of elasticity of a material. In reference to skin or human tissue, especially in cosmetic plastic surgery and dermatology, it is most commonly referred to as tissue firmness or tissue tightness. Changes in the firmness or tightness of tissue will result in changes to the slope of this linear portion of the stress-strain curve.

In one embodiment, a skin elasticity measurement device 60 may be used in system 1 to provide measurements of the skin firmness or elasticity of a tissue surface affected by fat volume removal and/or the coagulation of a subcutaneous tissue plane of interest as a feedback signal to a processor in generator 50 or in applicator 10, such that a desired skin elasticity or firmness can be achieved during a skin tightening procedure performed using applicator 10.

Referring to FIG. 1E, an exemplary method 150 for the treatment of lax soft tissue using an electrosurgical system including plasma energy device, such as system 1, is shown in accordance with an embodiment of the present disclosure. In step 152, before any treatment of patient tissue is performed, the skin elasticity, e.g., the modulus of elasticity, of a portion of a patient skin surface is measured using a skin elasticity measurement device 60 to establish a baseline for the skin elasticity of the portion of the skin surface. In step 154, a skin elasticity altering procedure (e.g., liposuction) is performed on a subcutaneous tissue plane of interest of the patient beneath the portion of the skin surface. In step 156, the skin elasticity of the portion of the skin surface is measured again using the skin elasticity measurement device 60. The measurements of steps 152 and 156 may be used to determine the change in skin elasticity resulting from the procedure performed in step 154. In step 158, a plasma beam is generated by applicator 10 (e.g., in the manner described above in relation to steps 106-110) in a single pass or predetermined number of passes over the subcutaneous tissue plane of interest to coagulate tissue and thereby tighten the portion of the skin surface.

In step 160, the skin elasticity of the portion of skin surface is measured again and, in step 162, it is determined (e.g., by a processor in generator 50) if the skin elasticity measurement from step 160 is within a threshold value. In one embodiment, the threshold value is based on the skin elasticity measurement obtained in step 152 (e.g., the skin elasticity of the patient before the procedure of step 154 is performed). If it is determined that the skin elasticity is not within the predetermined threshold, in step 162, applicator 10 is used to apply plasma in a second pass or predetermined number of passes over the subcutaneous tissue plane of interest, in step 158, and steps 158-162 are repeated until the skin elasticity of the portion of the skin surface is within the predetermined threshold. Alternatively, if it is determined that the skin elasticity is within the predetermined threshold, in step 162, method 164 ends as the portion of the skin surface is deemed to have the desired skin elasticity or firmness. For example, the threshold value may be the baseline value obtained in step 152; where if the value obtained in step 160 is greater than the value obtained in step 152, it is determined that the portion of the skin surface has become tightened. In this example, the modulus of elasticity determined in step 152 is compared to the modulus of elasticity determined in step 160. As another example, the threshold value may be the baseline value plus a predetermined offset; where if the value measured in step 160 is greater than the baseline value plus the offset, skin tightening has been achieved.

FIG. 1F illustrates exemplary results of the method 150 performed in accordance with the present disclosure. The values were obtained in conjunction with a liposuction treatment and subsequent passes of a plasma device of the present disclosure. FIG. 1F represents a graph of mean tissue firmness values, i.e., mean modulus of elasticity values, for each stage of treatment. For example, data point 170 was obtained by measuring skin firmness before the liposuction treatment, as in step 152 above, to obtain the baseline; data point 171 was obtained after liposuction was performed, as in step 154; and data points 172, 173, 174, 175, 176 and 177 were obtained after each pass of the plasma device. FIG. 1F demonstrates an immediate decrease in tissue firmness following liposuction as the fat volume is removed and tissue laxity increases. With each subsequent pass of helium plasma treatment, an increasing linear trend in tissue firmness is evident. The summary statistics of the change in the tissue firmness values for each stage of treatment compared to the previous stage of treatment is presented above in Table 1:

| Stage of Treatment | Mean Difference (SD) | |
|---|---|---|
| Comparison | g/in | |
| Pre-Lipo − Post-Lipo | −32.0 | (15.0) |
| Pass 1 − Post-Lipo | 13.1 | (5.5) |
| Pass 2 − Pass 1 | 11.7 | (4.8) |
| Pass 3 − Pass 2 | 10.6 | (3.9) |
| Pass 4 − Pass 3 | 9.4 | (4.3) |
| Pass 5 − Pass 4 | 12.6 | (4.3) |
| Pass 6 − Pass 5 | 11.4 | (7.0) |

The average decrease in tissue firmness as a result of liposuction was 32.0 g/in, i.e., grams of force/inches of distraction. So basically, it requires 32 grams of force to distract the tissue one inch. In this specific example, the amount of force it took to distract the tissue one inch decreased by 32 grams after performing liposuction. On average, a single treatment pass with the helium plasma energy device increased the firmness of the tissue by 11.5 g/in. Therefore, on average, the treated tissue began to exceed its pre-liposuction firmness value by the third treatment pass. Passes four through six served to increase the tissue firmness over and above the pre-liposuction value.

In some embodiments, the processor 51 in generator 50 may be configured to provide an alert (e.g., an alarm sound via alert 58 and/or a display notification via 10 interface 56) if it is determined that the skin elasticity is within the predetermined threshold, in step 162. In some embodiments, the processor 51 in generator 50 may be configured to adjust the waveform applied to the electrode in applicator 10 based on the amount the skin elasticity is above the predetermined threshold, in step 162. Additionally or alternatively, in some embodiments, the processor 51 in generator 50 may adjust the waveform based on the amount the skin elasticity measured in step 156 is above the predetermined threshold. In either case, a mapping between the amount of adjustment necessary to the waveform (e.g., in intensity, frequency, duration of pulses, etc.) as a function of the difference between the currently measuring elasticity (e.g., in step 156 or step 160) and the predetermined threshold may be stored in a memory 61 of generator 50 and used by the processor 51 in generator 50 to make adjustments to the waveform. It is to be appreciated that the predetermined threshold may be derived from the baseline measurement taken in step 152 or may be a value entered by an operator into the 10 interface 56 of the ESU 50.

It is to be appreciated that in some embodiments, step 152 and 154 may be removed from method 150 and steps 156-164 may be performed alone without the performance of a skin elasticity altering procedure.

The present disclosure provides various skin elasticity measurement devices, which may be used in system 1 and in the method 150 described above.

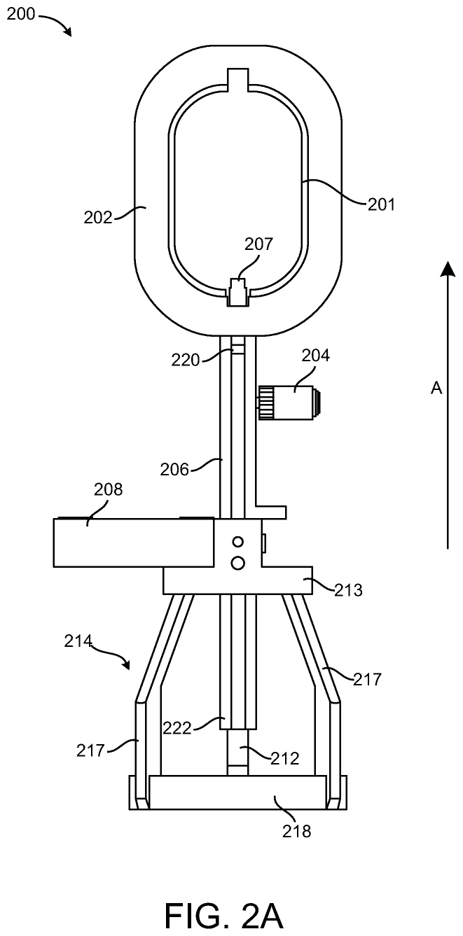

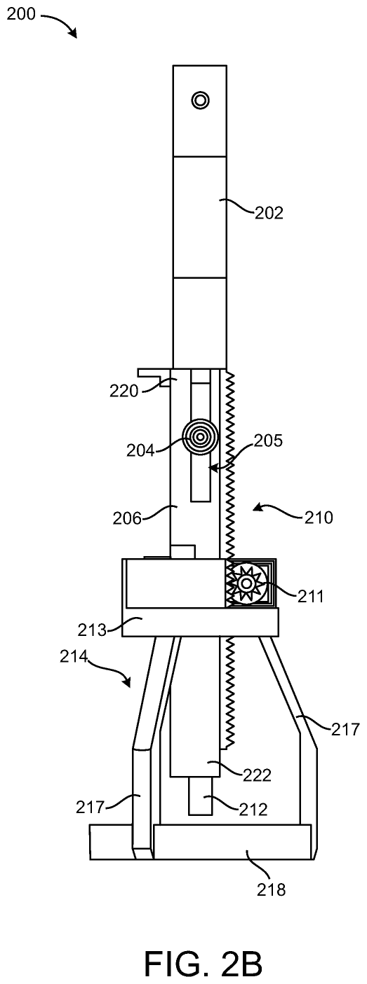

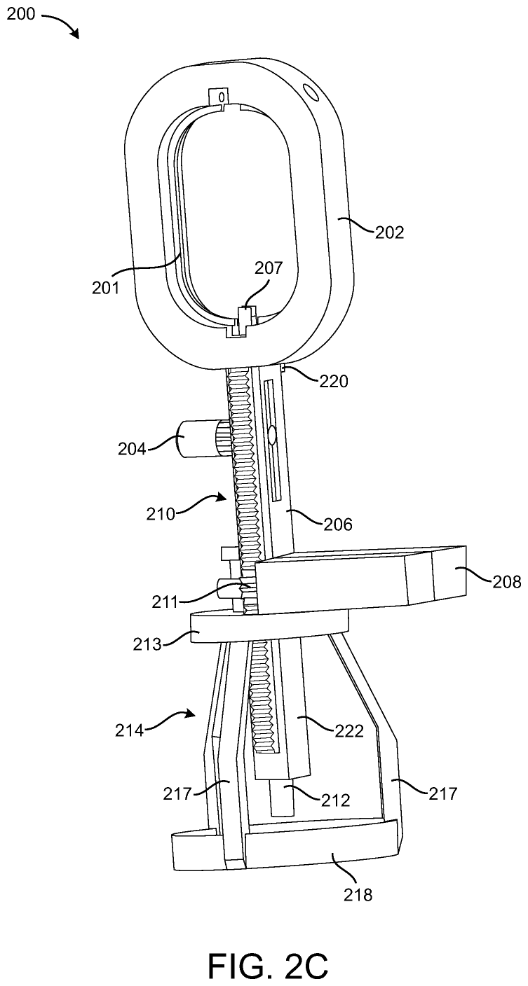

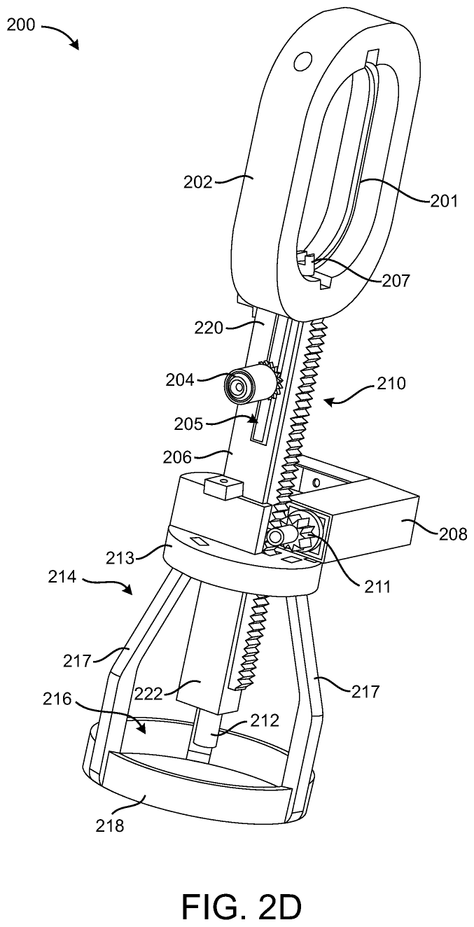

For example, referring to FIGS. 2A-2D, a skin elasticity measurement device 200 configured as a motorized tensile testing device is shown in accordance with an embodiment of the present disclosure. Device 200 includes a handle 202, a height adjuster 204, a shaft 206, a gauge coupler 207, a motor 208, a gripping member 212, and base 214.

Shaft 206 includes opposite ends 220, 222 and a linear set of gear teeth 210 extending along the length of shaft 206. Handle 202 includes an inner circumference or rim 201 and is coupled to end 220 of shaft 206. A skin gripping member 212 is disposed in and extends from end 222 of shaft 206. Skin gripping member 212 is coupled within shaft 210 to a gauge coupler 207, the gauge coupler 207 being disposed through an aperture in handle 202 and end 220 of shaft 206. Adjuster 204 is disposed through a slot 205 aligned along the linear length of shaft 206 (i.e., along direction A, as shown in FIG. 2A). Within shaft 206, adjuster 204 is coupled to gripping member 212, such that the distance that gripping member 212 extends from end 222 of shaft 206 is adjustable by sliding adjuster 204 along slot 205. Circumference 201 is dimensioned to receive a force or strain gauge having a complimentary shape to circumference 201, such that the force gauge is mounted to handle 201. In this way, handle 202 is configured both as a handle to be gripped and as a housing or mounting component for the force gauge. The force gauge is coupled to coupler 207, such that when a force is applied to gripping member 212 in a direction opposite to direction A, the force is transferred to coupler 207 and measured by the force gauge.

Base 214 includes a mounting component 213, posts 217 and a ring 218, where ring 218 defines an aperture 216. Ring 218 is coupled to mounting component 213 via posts 217. Shaft 206 is slidably disposed through an aperture of mounting component 213 and extends into an inner space contained between posts 217. Motor 208 is mounted to mounting component 213. Motor 208 includes a gear 211 having a set of gear teeth configured to interact with gear teeth 210 to extend and retract shaft 206 relative to base 214 when gear 211 is rotated. When gear 211 is rotated by motor 208 in a first direction, shaft 206 extends away from base 214 in a direction A (as shown in FIG. 2A), and when gear 211 is rotated by motor 208 in a second direction, shaft 206 is drawn toward base 214 (i.e., opposite to direction A).

In use, ring 218 is placed against patent skin and gear 211 is rotated in the second direction to extend gripping member 212 through aperture 216. In one embodiment, gripping member 212 is configured to grip the skin surface of a patient directly. In another embodiment, gripping member 212 is configured to attach to an interface (as will be described below) that is attached to the skin surface. In either case, after gripping member 212 is coupled to patient skin surface (either directly or indirectly), height adjuster 204 may be advanced through slot 205, such that the skin coupled to gripping member 212 achieves a desired initial tension (e.g., as measured by the force readings of the force gauge). Then, gear 211 is rotated in the first direction to retract gripping member 212 away from ring 218 and the patient skin in direction A, thus pulling a portion of the patient skin disposed within the outer circumference of ring 218 in direction A. In one embodiment, gear 211 is rotated by motor 208 at a constant rate, such that the portion of the skin being pulled by gripping member 212 is pulled to a desired displacement. The force gauge is calibrated to the displacement of gripping member 212, therefore, based on the force measured by the force gauge, the displacement of gripping member 212 can be determined. The force gauge is used to measure the force at various displacements of gripping member 212. The measured forces may then be provided to a processor 51 of generator 50 and used in method 150 described above to determine the skin elasticity. For example, the measured forces, i.e., a force at a particular displacement, may be transmitted from the force gauge to the generator 50 via the communication module 66 via hardwired or wireless means. Alternatively, the measured forces may be entered into the generator via the 10 interface 56, e.g., a touchscreen. The processor 51 of the generator 50 may then use the measured forces to determine the modulus of elasticity at different points in a treatment procedure as described above.

Although a motor 208 is used in device 200 to extend or retract shaft 206 to displace or pull a skin surface, in another embodiment, shaft 206 may be extended or retracted using an electromagnetic mechanism. For example, shaft 206 may include a magnet and device 200 may further include an electromagnet, used to change the magnetic force between the electromagnet and the magnet included on the shaft 206. In this way, by selectively controlling the magnetic force between the electromagnet and the magnet of the shaft, shaft 206 can be extended or retracted to the desired displacement.

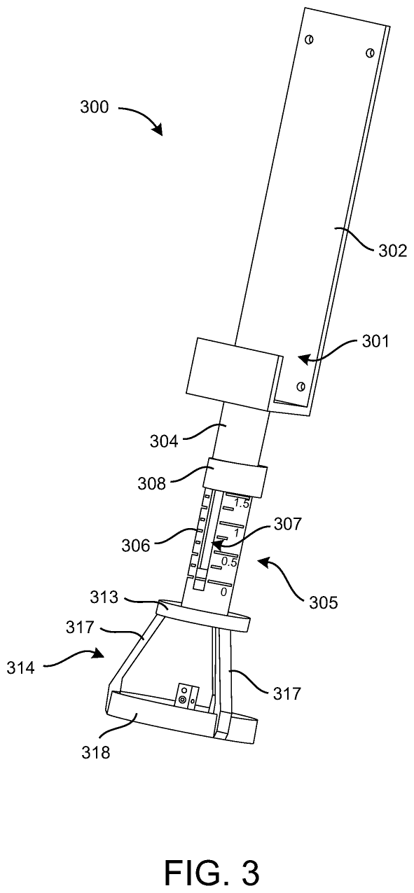

In another embodiment, the skin elasticity measurement device may be configured as a manually operated tensile testing device. For example, referring to FIG. 3, a skin elasticity measurement device 300 is shown in accordance with an embodiment of the present disclosure.

Device 300 includes a handle 302, first and second tubular members 304, 306, height adjuster 308, mounting component 313, and base 314. A first end of tubular member 304 is coupled to handle 302 and a second end of tubular member 304 is disposed through a first end of tubular member 306, such that tubular members 304, 306 are slidable with respect to each other. Tubular member 306 includes a transparent slot 307, which shows the position of the second end of tubular member 304 disposed within tubular member 306. Tubular member 306 further includes a plurality of markings 305, which indicate the position and displacement of the second end of tubular member 304 within the interior of tubular member 306. Height adjuster 308 is coupled to tubular member 306 at a desired position and secured to the desired position along member 306 to limit the range of motion tubular members 304, 306 can slide with respect to one another. A second end of tubular member 306 is coupled to a mounting component 313. Base 314 includes a plurality of posts 317 and a ring 318, where posts 317 are coupled to mounting component 313 and ring 318. It is to be appreciated that, since tubular members 304, 306 are slidably with respect to each other, ring 318 and handle 302 are extendable and retractable with respect to each other.

Handle 302 is configured both as a handle to be gripped by a user and as a mounting component for a force or strain gauge. Handle 302 includes a slot 301 configured to receive the force or strain gauge. The slot 301 includes an aperture that makes accessible the interiors of tubular members 304, 308. A hook of the force gauge is disposed in the aperture and attached to a first end of a string or other coupling member which is disposed through the interiors of tubular members 304, 308. An adhesive, suture loop, or other gripping means may be used to grip a portion of a skin surface. A second end of the string is attached the suture loop, adhesive, or other gripping means. In this way, after attaching the string or coupling member to the gripping means, handle 302 is pulled in a direction away from ring 318 to increase the tension in the string and pull a portion of the skin surface toward handle 302. The displacement of the pulled skin surface is measured by observing the position of the second end of tubular member 304 with respect to the markings 305. The force for a given displacement is measured by the force gauge. In this way, using the force measured for each given displacement, the skin elasticity of the portion of the skin surface may be determined (e.g., the modulus of elasticity may be determined by a processor 51 in generator 50) and used in the method 150 described above. For example, the force measurements may be manually entered into the ESU 50 via 10 interface 56, e.g., a touchscreen, or electronically transmitted to the communication module 66 of the ESU 50 which then provided to processor 51.

It is to be appreciated that in devices 200 and 300 shown in FIGS. 2-3, the gripping means or member for gripping patient skin surface may be any interface configured to be attached to or grip the patient skin surface and also be attached to a force gauge. For example, in one embodiment, the interface may be suture loops placed through the dermis/skin, where the suture loops may be attached to the force gauge, e.g., via the gripping member and force coupler. In another embodiment, the interface may include a pad having an adhesive backing and a hook, loop, or other means for enabling the adhesive backed pad to be attached to the gripping member. Alternatively, the glue may be used to grip a side of the pad to the skin surface. In another embodiment, an adhesive backed pad may be used for gripping the skin surface and suture loops may be threaded through both the pad and the dermis/skin, where the loops may then be attached to the force gauge or the gripping member. It is to be appreciated that the adhesive backed pad may be configured in a similar manner to ECG pads, steri-strips, or other adhesively backed pads.

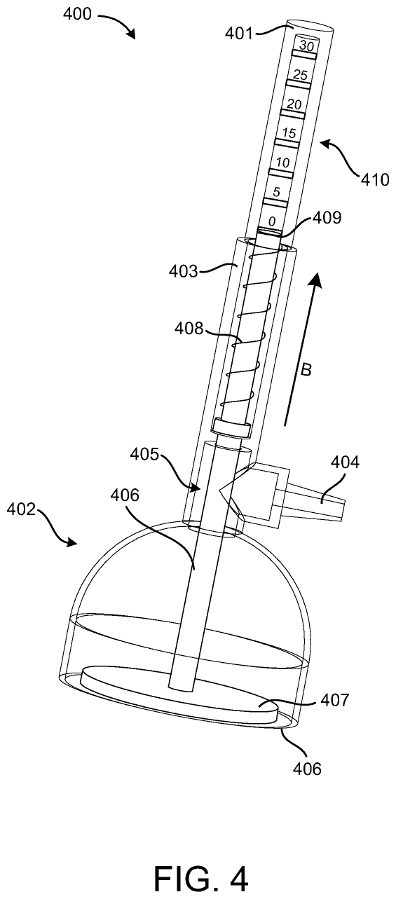

In another embodiment, the skin elasticity measurement device may be configured as a vacuum assisted device. For example, referring to FIG. 4, a skin elasticity measurement device 400 is shown in accordance with an embodiment of the present disclosure. Device 400 includes housing 401, a spring 408, and an inner measuring component 405. Housing 401 includes a port 404, a tubular portion 403 and a base 402 configured as a suction cup. Base 402 includes a working face or rim portion 406 and a hollow inner cavity (defined by the dome shape of base 402). Component 405 includes a stem 406 slidably disposed through the interior of tubular member 403. Component 405 further includes a disc 407 coupled to the stem 406 and disposed within base 402. Spring 408 is disposed around a portion of stem 406 and configured to bias component 405 toward rim 406. Housing 401 includes a plurality of markings 410.

Port 404 is configured to be connected to a vacuum or negative air pressure device. When rim 406 is placed on the skin of a patient, the interior of base 402 is sealed and the vacuum or negative air pressure device is used to create a pressure difference between the inner cavity of base 402 and the atmospheric pressure. The pressure difference displaces a portion of the skin that portion 406 is disposed over in a vertical direction B (i.e., toward the inner cavity of base 402). The displacement of the skin in the direction B forces component 405 to also be displaced in the direction B. The position of the tip 409 of stem 406 with respect to markings 410 indicates the displacement of the portion of the skin drawn into the interior of base 402. The displacement of the skin is proportional to the pressure difference between the inner cavity and the atmosphere. The displacement is then recorded and can be provided to the generator unit 50 to determine the skin elasticity and used in method 150. In one embodiment, since the pressure is constant, the effectiveness of the elasticity altering procedure may be determined by comparing changes in the measured displacement from the measured baseline value. For example, if a tissue elasticity altering procedure is effective, the displacement of tissue after the procedure should be less than the baseline measurement. In another embodiment, the modulus may be calculated by determining the force and using the measured displacement. In this embodiment, since the pressure and the diameter of the disc 407 is known, force may be calculated using the formula: pressure=force/area. A baseline modulus may then be compared to a modulus calculated after a tissue elasticity altering procedure.

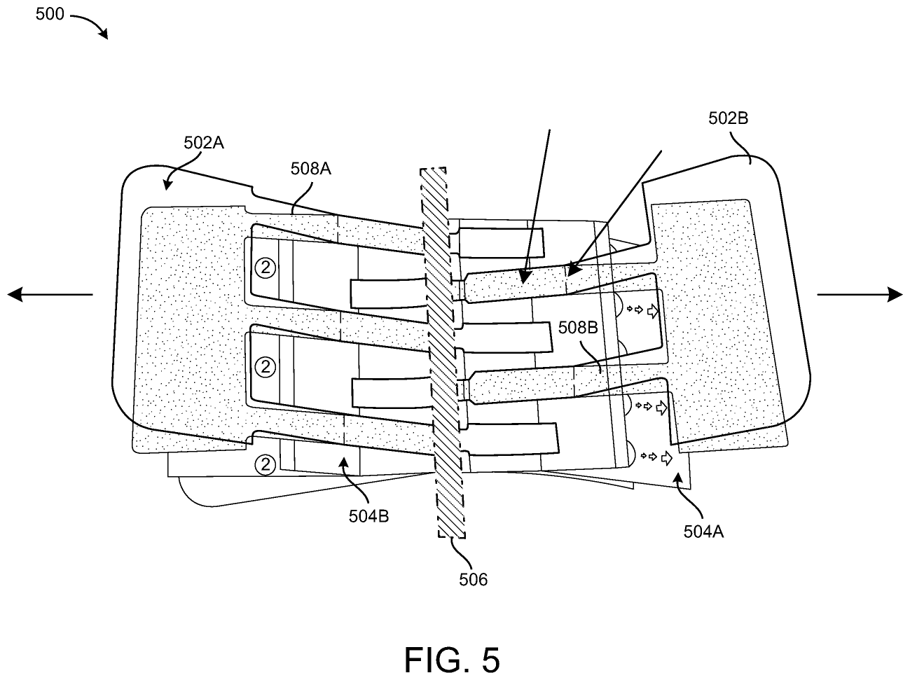

In another embodiment, the skin elasticity measurement device may be configured as a lateral/shear measurement device. For example, referring to FIG. 5, a skin elasticity measurement device 500 is shown in accordance with an embodiment of the present disclosure. Device 500 includes pulling tabs 502A, 502B, and adhesive tabs 504A, 504B. Tabs 504A, 504B include adhesive backing, such that tabs 504A, 504B can be placed on and adhered to the surface of a patient's skin. A central portion 506 separating tabs 504A, 504B is configured without any adhesive. Pulling tab 502A is attached via attaching member 508A to adhesive tab 504A and pulling tab 502B is attached via attaching members 508B to adhesive tab 504B. In this way, when adhesive tabs 504A, 504B are placed on and adhered to the surface of the patient's skin, tabs 502A, 502B may be pulled in a direction away from each other to cause adhesive tabs 504A, 504B to be pulled in a direction toward each other. When adhesive tabs 504A, 504B are pulled toward each other, a portion of the patient's skin adhered to by tab 504A and a portion of the patient's skin adhered to by tab 504B (i.e., the skin on either side of central portion 506) are pulled toward each other. In one embodiment, a gauge is used by a user to pull tabs 502A, 502B and thus bring tabs 504A, 504B together, i.e., a separate force gauge is coupled to each of pull tabs 502A, 502B. The force required to bring tabs 504A, 504B together is determined by combining the force measurements of the gauges coupled to each of the tabs 502A, 502B. The force measured by the gauges is proportional to the skin elasticity of the skin underneath and between tabs 504A, 504B. The force, and optionally the displacement of the tabs 504A, 504B, may then be provided to the generator 50, e.g., either electronically transmitted or manually entered, to determine skin elasticity and used in method 150. In one embodiment, the determined force and displacement of the tabs are employed by the generator 50 to determine the modulus of elasticity. In other embodiments, the generator 50 may compare relative force values to determine the effectiveness of a procedure, i.e., comparing the force measurements before and after a procedure.

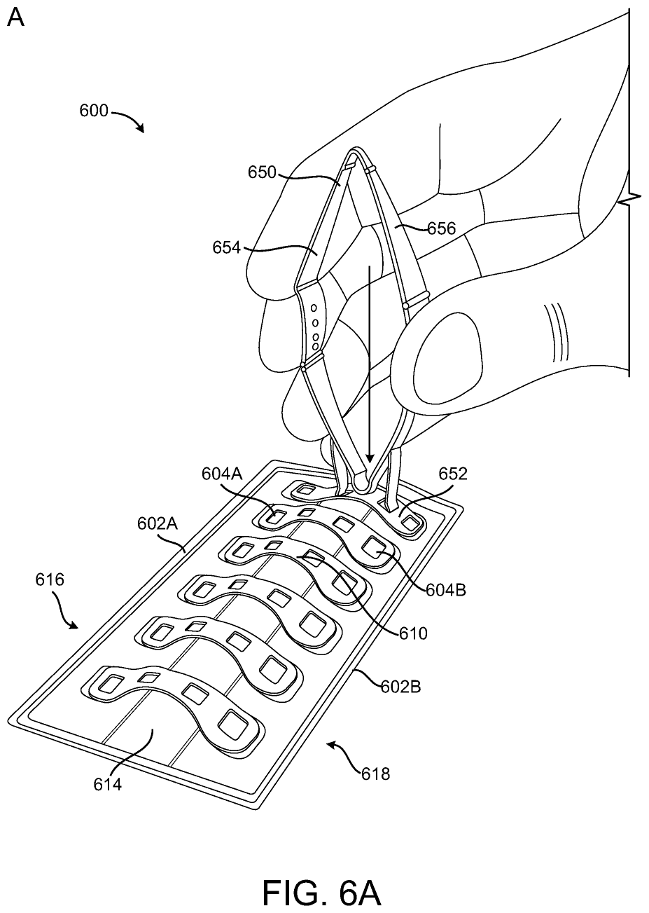

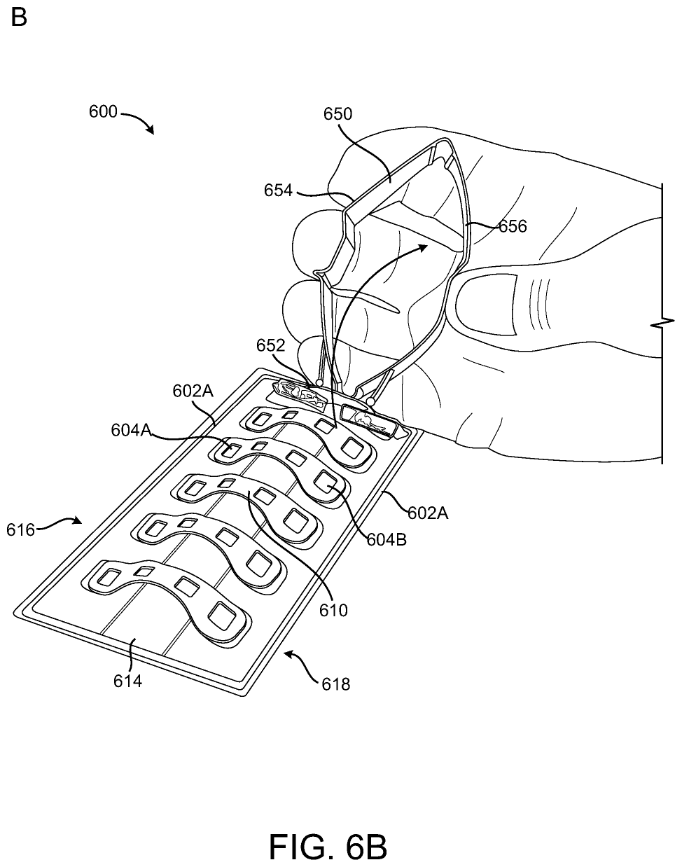

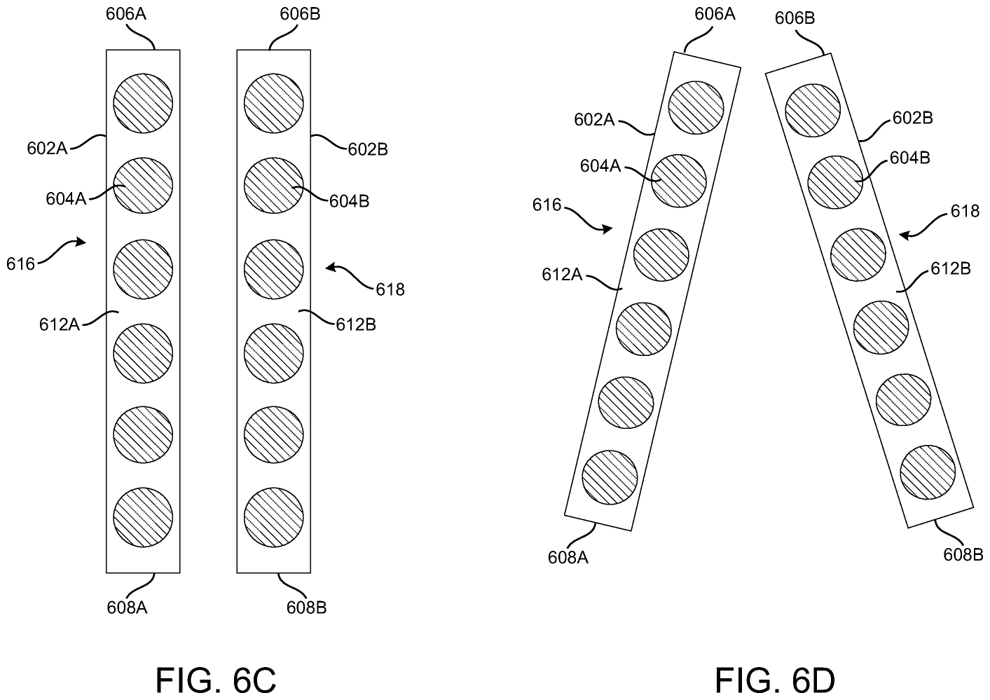

In another embodiment, the skin elasticity measurement device may be configured as a pincher/lateral load measurement device. For example, referring to FIGS. 6A-6D, a skin elasticity measurement device 600 is shown in accordance with an embodiment of the present disclosure. Device 600 includes linear strips 602A, 602B, which are disposed on sides 616, 618, respectively of device 600 and connected by a plurality of equally spaced connection members 610. Each strip 602 includes a first side or surface 612, a second side or surface, opposite to the first side (not shown), and ends 606, 608. Side 612 of each strip 602 includes a plurality of equally spaced protrusions or tabs 604. In one embodiment, corresponding pairs of tabs 604A, 604B extend through connection members 610. The second side of each strip 602 includes adhesive backing, such that the second side or surface of each strip 602 can be adhered to the skin of a patient. In one embodiment, a central section 614 (as shown in FIGS. 6A and 6B) is disposed between and connects strips 602A, 602B and extends from ends 606A, 606B to ends 608A, 608B. The central section 614 is configured to be flexible and does not include an adhesive backing such that strips 602A, 602B can be pulled toward each other when strips 602A, 602B are attached to patient skin. In another embodiment, as show FIGS. 6C, 6D, central section 614 may be eliminated and strips 602A, 602B may be separate and distinct components.

As shown in FIG. 6C, in one embodiment, device 600 is configured such that end 606A and end 608A are separated by the same distance as ends 608A, 608B. In this way, in the embodiment shown in FIG. 6C, each corresponding pair of tabs 604A, 604B are equidistantly spaced. As shown in FIG. 6D, in another embodiment, device 600 is configured such that ends 606A, 606B are disposed at a different distance than ends 608A, 608B. In this way, in the embodiment shown in FIG. 6D each corresponding pair of tabs 606A, 606B are spaced at a different distance from each other. It is to be appreciated that in each of the embodiments shown in FIGS. 6C and 6D the distance between each corresponding tab pair 606A, 606B is known.

In use, when strips 602A, 602B are adhered to patient skin, a device, such as device 650 (e.g., forceps) is used to grip a pair of tabs 604A, 604B to bring the pair of tabs 604A, 604B together. In one embodiment, device 650 includes tong arms 654, 656 and a gripping member 652. Gripping member 652 includes respective slots or apertures configured to receive a pair of tabs 604A, 604B. Arms 654, 656 are coupled to gripping member 652 and when pinched by a user, are configured to cause the slots or apertures containing tabs 604A, 604B to be pulled together, thus also pulling the pair of tabs 604A, 604B together. When the pair of tabs 604A, 604B are brought together, the skin between tabs 604A, 604B is pinched and the skin on either side 616, 618 of device 600 is pulled toward device 600. A force gauge (or load cell) may be coupled to arms 654, 656 to measure the amount of force required to pull the pair of tabs 604A, 604B to within a predetermined distance of each other, i.e., so that when the arms 654, 656 are squeezed together the arms 654, 656 places a load on either side of the force gauge/load cell. Measurements for the force may be recorded for various displacements of tabs 604A, 604B with respect to each other and provided to generator 50, via electronic transmission or manually, to generate a displacement vs. load curve. Thereafter, the slope of the generated displacement vs. load curve can be calculated by the generator 50. The slope of the curve represents the skin elasticity and may be used by generator 50 in method 150, as described above.

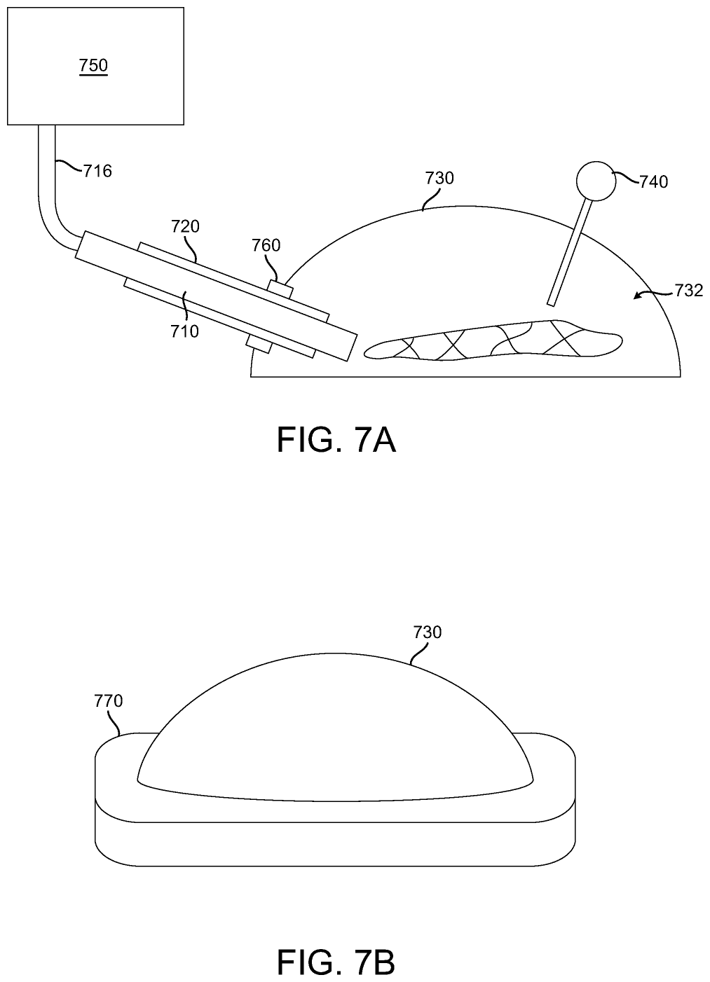

In another embodiment, skin elasticity may be measured by monitoring the back pressure generated by gasses introduced percutaneously to a tissue plane of interest. For example, referring to FIG. 7A, an electrosurgical generator unit and gas compressor (ESU) 750 is shown coupled to a plasma generator or applicator 710 via a cable 716. ESU 750 is configured to provide electrosurgical energy and at least one gas (e.g., an inert gas, such as, but not limited to, helium) via cable 716 to applicator 710. Applicator 710 is configured to apply a plasma beam generated using the electrosurgical energy and at least one gas to a tissue plane 732 below the tissue or skin surface 730. In this way, the tissue plane may be treated during a skin tightening or firming procedure. The applicator 710 is introduced to the tissue plane 732 using a cannula 720.

In the embodiment shown in FIG. 7A, a pressure sensor 740 may be introduced subcutaneously into the tissue plane 732. Sensor 740 is configured to measure pressure in the tissue pane 732 and provide the pressure measurements to ESU 750 (e.g., via a hardwired or wireless communication link or connection). In one embodiment, pressure sensor 740 may be disposed on the distal end of applicator 710 and coupled to ESU 750 via cable 716. During a period of time where plasma is not being applied to the tissue plane 732, ESU 750 is configured to provide pulses of the at least one gas at a predetermined amplitude and frequency to applicator 710 and applicator 710 is configured to introduce the pulses of gas into the tissue plane 732. Back pressure of the tissue in the tissue plane 732 resisting being inflated by the pulses of gas introduced is generated when the pulses of gas are introduced into the tissue plane 732 and the back pressure is measured by sensor 740. The back pressure varies depending on the firmness of the skin surface 740 and connective tissue below the skin surface 740, e.g., the back pressure increases as the firmness increases, i.e., skin is tightened. Generator 750 is configured to determine the skin elasticity of skin surface 730 by comparing how the amplitude and frequency of the back pressure varies from the predetermined amplitude and frequency of the introduced pulses. It is to be appreciated that generator 750 and applicator 710 may be used to perform method 150, described above. For example, in one embodiment, gas is introduced into the tissue plane of interest 732 and the back pressure is measured to obtain a baseline measurement. A tissue altering procedure is performed and gas is then introduced again to get a post-treatment measurement. If the post-treatment measurement shows that the back pressure is greater than the back pressure measurement of the baseline, tissue firmness has increased.

In one embodiment, a seal 760 is used around the port where the cannula 720 is introduced. The seal 760 may be configured to allow for complete or partial gas sealing of the tissue plane 732 of interest.

Referring to FIG. 7B, in some embodiments, a ring 770 of known dimensions may be placed on top of an area of the skin surface 730 to constrain the pulsed gasses introduced into the tissue plane 732 to a specific area of interest confined within the circumference of the ring.

It is to be appreciated that, in the embodiment of FIG. 7A, although ESU 750 is configured as both an electrosurgical generator and a compressor, in other embodiments a separate compressor may be used to provide gas (pulsed or otherwise) to applicator 710.

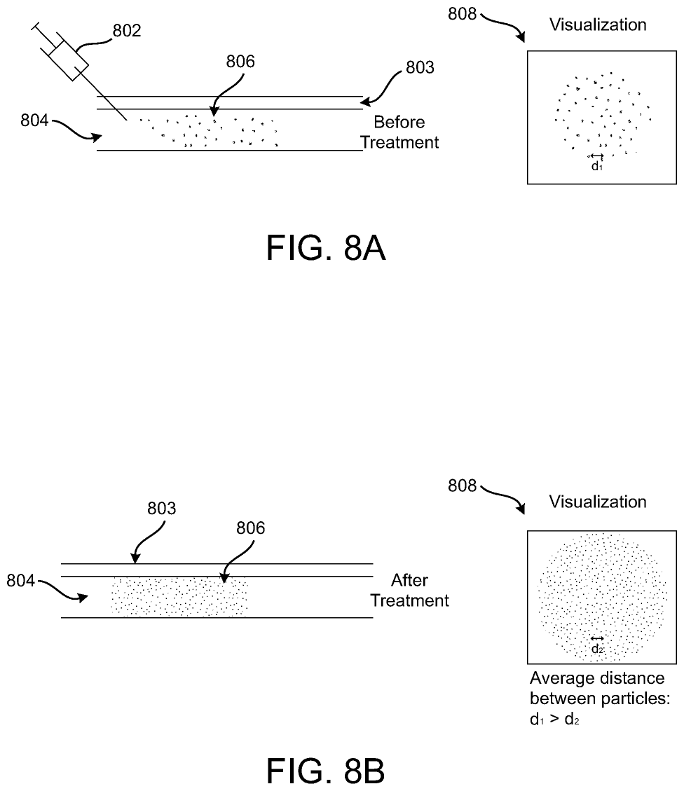

Referring to FIGS. 8A and 8B, in another embodiment, the skin elasticity of a tissue surface may be determined based on the changes to the density of a contrast fluid injected into the tissue plane. For example, referring to FIG. 8A, before a procedure or treatment (e.g., liposuction and/or application of plasma) is performed on the subcutaneous tissue plane or adipose tissue 804, contrast fluid 806 is injected through the tissue surface or skin 803 into tissue 804 using a syringe or other injection device 802. It is to be appreciated that the contrast fluid may include particles in the fluid, e.g., gold nanoparticles, to determine a density or other baseline value of the fluid. Various visualization techniques 808 (e.g., x-rays, sonography, etc.) configured to detect the contrast fluid amongst the tissue 804 can be used to determine the spacing and/or density of the contrast fluid and/or particles before the procedure or treatment is performed. Referring to FIG. 8B, after the procedure or treatment is performed, the visualization techniques 808 can be used be used again to determine the spacing and/or density of the contrast fluid after procedure or treatment is performed. The skin tightness or elasticity of skin 803 can by determined by comparing the change in spacing/density of the particles of contrast fluid 806 before and after the treatment. For example, if the density of the particles after treatment (d2) is greater than the density of the baseline or pretreatment (d1), tissue firmness has increased. The process of determining the spacing and/or density of the contrast fluid can be repeated throughout various stages of a procedure or treatment. It is to be appreciated that the spacing and/or density of the contrast fluid may be determined using imaging software configured to employ imaging techniques 808. The skin tightness or elasticity measurements obtained by comparing the changing spacing/density of the contrast fluid 806 is then provided to generator 50 and may be used in the method 150, described above.

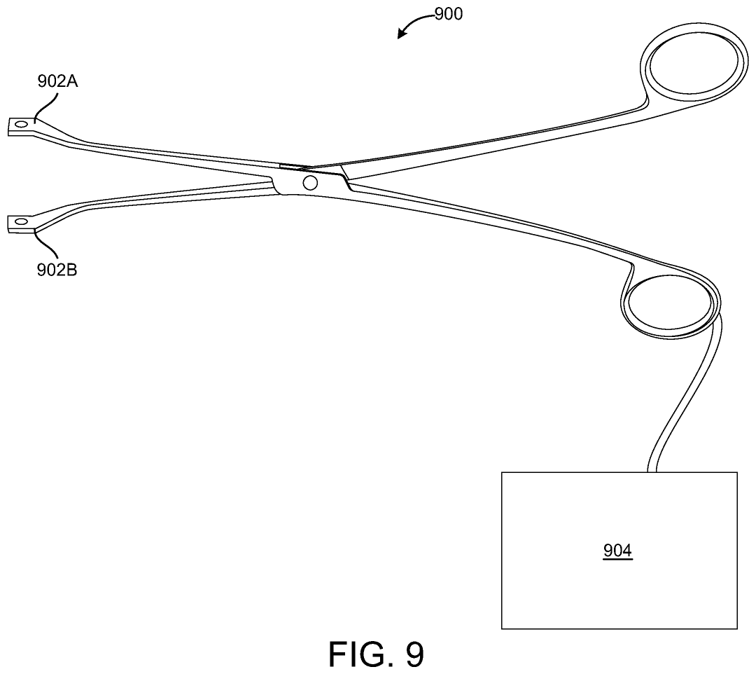

Referring to FIG. 9, in another embodiment according to the present disclosure, the skin elasticity measurement device may be configured as pressure sensitive forceps 900. Forceps 900 include pressure sensitive jaws or gripping members 902A, 902B disposed on distal ends of forceps 900. Jaws 902A, 902B are configured to grip a tissue surface and measure a pressure generated when the gripped surface is pinched between jaws 902A, 902B. The pressure measurements are provided via a cable or wireless means to a reader 904, which records the measured pressure for a specific displacement of the gripped tissue surface. In one embodiment, the pressure measurements are indicative of the force required to displace the tissue. It is to be appreciated that the distance between the jaws 902A. 902B when forceps 900 are in an open position is known. Thus, when jaws 902A, 902B grip a tissue surface from an open position and are brought together, the known distance between jaws 902A, 902B in the open position is used to estimate the displacement of the gripped tissue surface. Alternatively, the forceps 900 may include a sensor that measures and provides the distance between the jaws 902A, 902B at any position through the range of motion of the jaws 902A, 902B. The measured distance between the jaws 902A, 902B is indicate of the displacement of the tissue grasped. The measurements, i.e., pressure/force and/or displacement, may then be provided to generator 50 to determine skin elasticity and used in the method 150, described above. It is to be appreciated that the reader may be disposed in ESU 50 and the results may be displayed on the 10 interface 56 of the ESU 50.

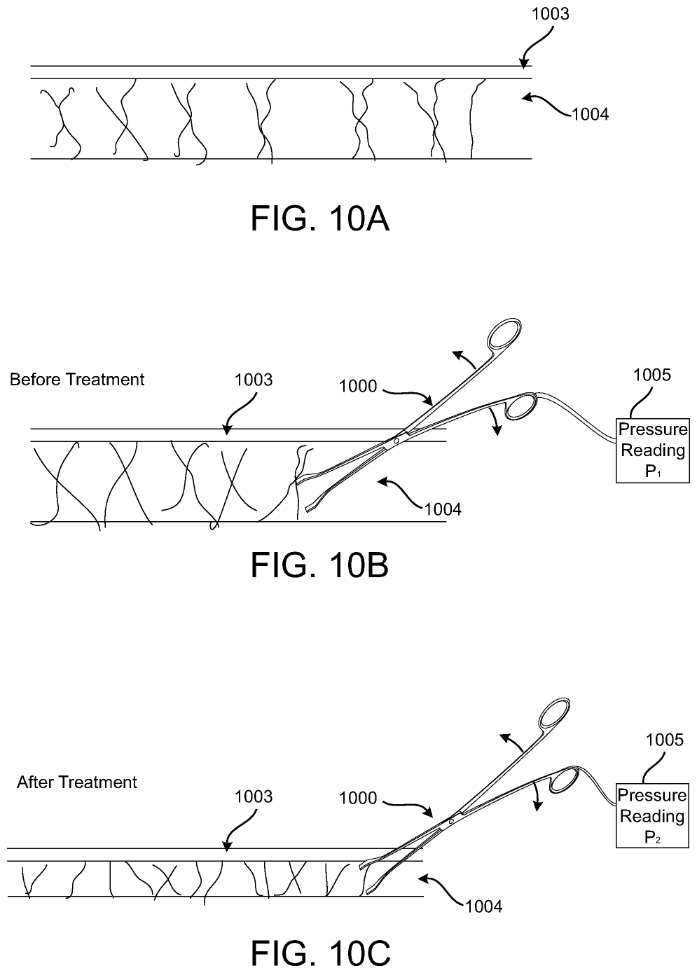

Referring to FIGS. 10A-10C, in another embodiment according to the present disclosure, the skin elasticity measurement device may be configured as a pressure sensitive clamp/forceps endoscope 1000. The device 1000 includes pressure sensitive tips configured to measure pressure or resistance when the tips are opened (moved away from each other) or closed (moved closer to each other). The measurements are provided to a reader 1005, which may then be provided to generator 50. The distal ends of device 1000 include the pressure sensitive tips are inserted percutaneously through the skin 1003 and into a tissue plane or adipose layer 1004 and the pressure sensitive tips are opened or moved away from each other within the plane or layer 1004. The resistance exerted by the tissue plane 1004 to the displacement of the tips of device 1000 is recorded by reader 1005 both before and after the treatment as indicated in FIGS. 10B, 10C. The measurements recorded by reader 1005 may be used in the method 150, described above. For example, the resistance at each recorded displacement may be equated to a predetermined force or stress, wherein the stress and displacement measurements may be further employed to determine the modulus of elasticity.

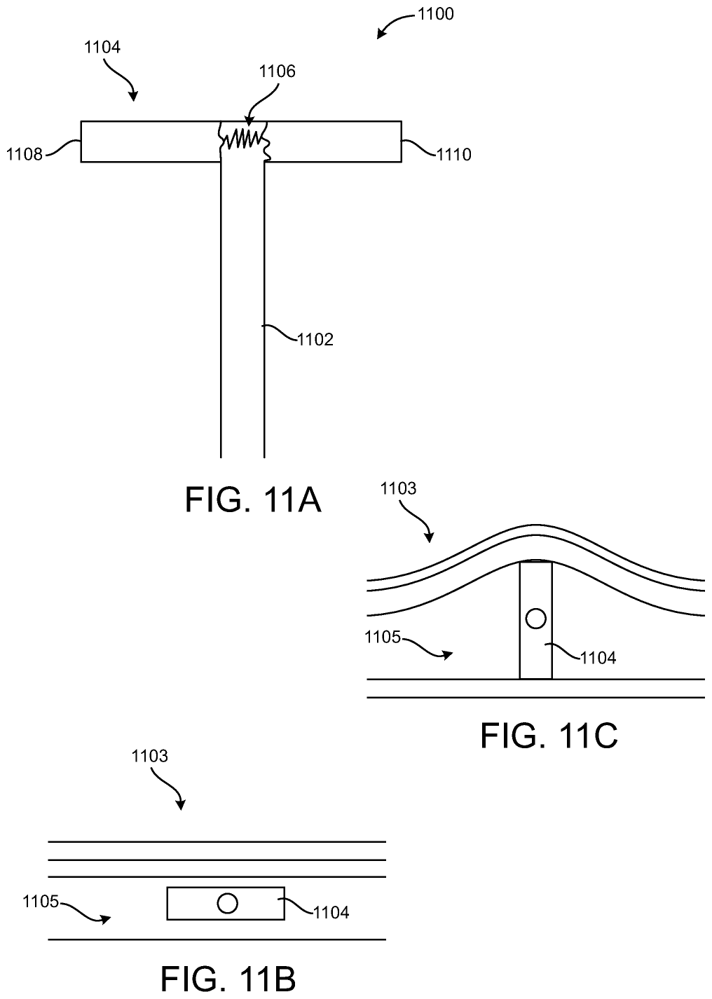

Referring to FIG. 11A, in another embodiment according to the present disclosure, the skin elasticity measurement device may be configured as t-rod shaped device 1100. The device 1100 includes a linear handle portion 1102 and a linear tip portion 1104, where an end of handle 1102 is coupled to the center of tip 1104 and tip 1004 extends perpendicularly relative to handle 1102. Tip 1104 may include one or more pressure sensors 1106 (e.g., disposed in a central portion as shown in FIG. 11A and/or disposed on the ends 1108, 1110 of tip 1104) configured to measure the pressure or external forces applied to tip 1104. Referring to FIG. 11B, the tip 1104 is inserted through skin 1103 into a subcutaneous tissue plane 1105 and rotated via handle 1102 until tip 1104 is approximately perpendicular to the tissue plane 1105 (as shown in FIG. 11C), thus separating two layers of tissue. The sensors 1106 in tip 1104 measure the pressure or force exerted on the tip 1104 by the tissue in plane 1105 contacted tip 1104. The measurements may be obtained before, after, and/or during a tissue tightening treatment or other procedure to monitor the changes in skin elasticity. The measurements obtained by the sensors 1106 may be provided to a reader and/or generator 50, such that the skin elasticity of skin 1103 may be determined based on the measurements (e.g., and used in method 150 described above). During the tissue tightening treatment, the tissue in plane 1105 shrinks and the measured pressure by tip 1104 increases, enabling the determination (e.g., by generator 50) of the change in the skin elasticity of skin 1103. Referring to FIG. 11C, in some embodiments, tip 1104 may be swiveled and/or rotated via handle 1104 into a desired position within the tissue plane 1105, e.g., perpendicular to the tissue plane 1105. It is to be appreciated that in the embodiments of FIGS. 11A-C the effectiveness of an elasticity altering procedure may be determined by comparing the relative pressure measurements before treatment and measurements taken after subsequent treatments, for example, subsequent passes of a plasma device.

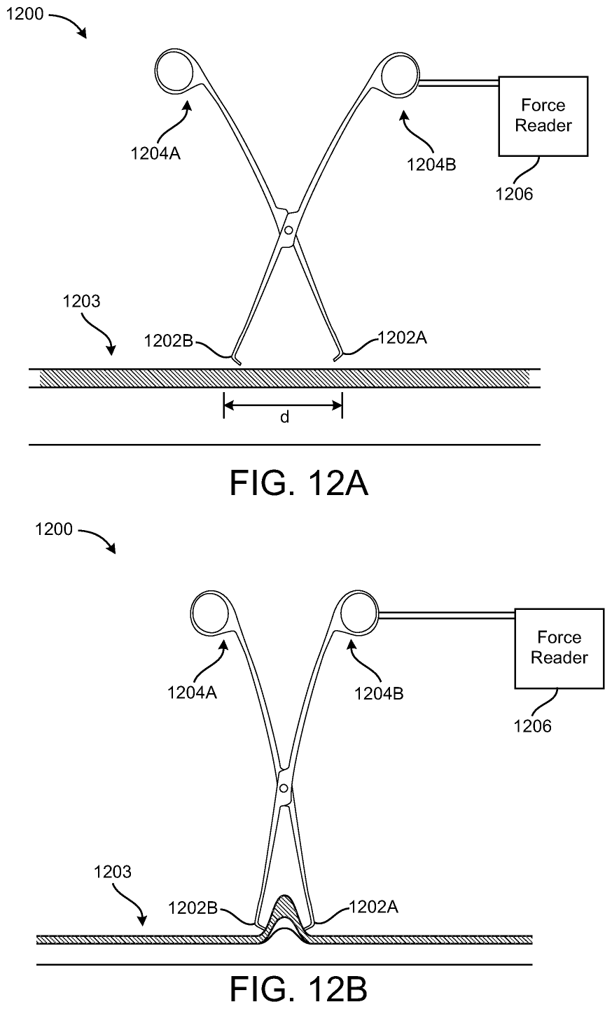

Referring to FIGS. 12A, 12B in another embodiment of the present disclosure, the skin elasticity measurement device may be configured as forceps 1200. Device 1200 includes a pairs of grasping members 1204A, 1204B. The distal end of grasping member 1204A includes load/pressure sensor 1202A and the distal end of grasping member 1204B includes load/pressure sensor 1202B. Device 1200 is shown with grasping members 1204A, 1204B in an open position in FIG. 12A and in a closed position in FIG. 12B. When grasping members 1204A, 1204B are in an open position, load/pressure sensors 1202A, 1202B are separated by a known predetermined distance d. The distal ends of grasping members 1204A, 1204B may be used to grasp a portion of skin 1203 and pinch the grasped portion of skin 1203 by bring the distal ends of grasping members together (as shown in FIG. 12B). The pressure sensors 1202A, 1202B are configured to measure the amount of force required to pull the grasped portion of skin 1203 together from the predetermined distance d and provide (e.g., via hardwired or wireless connection) the measurements to a force reader 1206 to be read, e.g., the force at a particular displacement. The measurements obtained by the sensors 1202A, 1202B may be provided to generator 50, such the skin elasticity of skin 1103 may be determined based on the measurements and used in method 150 described above. It is to be appreciated that the reader may be disposed in generator 50.

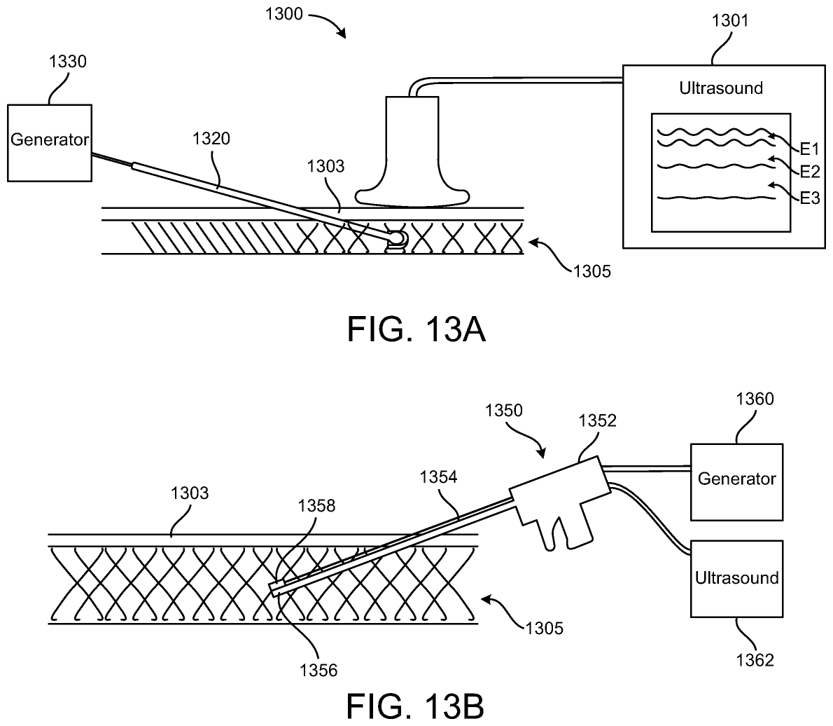

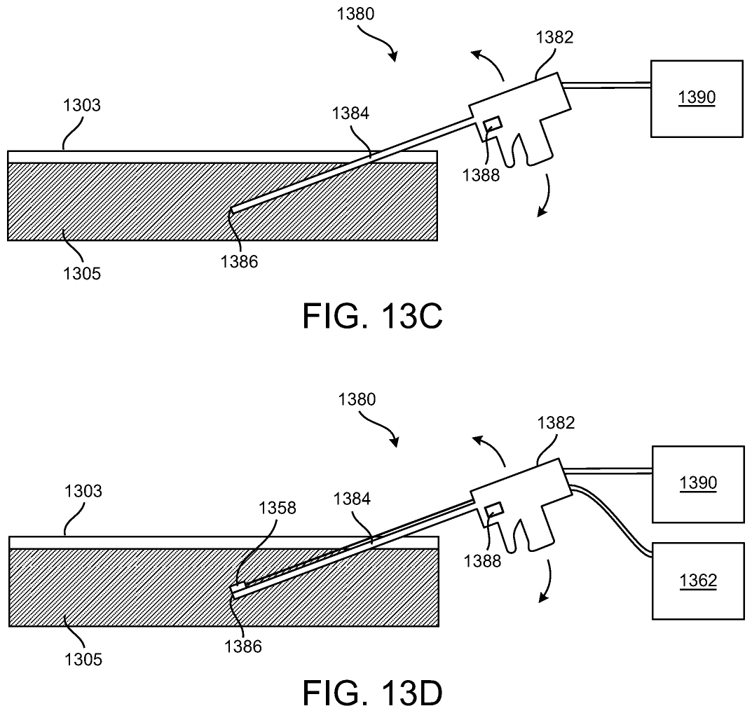

Referring to FIG. 13A in another embodiment of the present disclosure, the skin elasticity measurement device may be configured as an ultrasound device 1300. In this embodiment, device 1300 (e.g., a transducer) may be disposed adjacent to skin 1303 and used to introduce sound waves into subcutaneous tissue 1305 and measure returning sounds waves propagating through tissue 1305. The acoustic data measured by device 1300 is provided to a display and analysis module 1301. The data provided to module 1301 is used to detect the mechanical properties of cutaneous and/or subcutaneous tissue layers using ultrasound elastography or other imaging modalities. The acoustical data obtained by device 1300 may be used by module 1301 and/or by a processor in generator 50 to determine the skin elasticity of a portion of skin surface 1303. It is to be appreciated that module 1301 may be disposed in generator 50. Device 1300 may be used to obtain acoustical data before, during, and/or after a skin treatment (or elasticity altering) procedure is performed on skin 1303 and/or subcutaneous tissue 1305. The skin treatment may be liposuction and/or coagulation of tissue 1305 using a plasma applicator or handpiece 1320 coupled to a generator 1330.

In another embodiment, device 1300 may integrated into handpiece 1320. For example, referring to FIG. 13B, a plasma handpiece 1350 is shown in accordance with an embodiment of the present disclosure. Handpiece 1350 includes a handle housing 1352, a shaft 1354, and a transducer probe 1358 disposed on a distal end 1356 of shaft 1354. Handpiece 1350 is coupled to generator 1360 for receiving electrosurgical energy. Handpiece 1350 may receive an inert gas from generator 1360 or from a separate gas source. An electrode (not shown) is disposed in shaft 1354 and is configured to energize the inert gas provided to handpiece 1350 using energy received from generator 1360 to generate plasma that is ejected from distal end 1356 of shaft 1354 to treat tissue, such as tissue 1305. Handpiece 1350 is further coupled to ultrasound display and analysis module 1362, such that, acoustical data sensed by transducer 1358 is provided to module 1362 for analysis and display. It is to be appreciated that one or more wires for carrying acoustical data couple transducer 1358 to module 1362. It is to be appreciated that in some embodiments, ultrasound display and analysis module 1362 may be disposed in generator 1360. The one or more wire extend from module 1362 into housing 1352 and along the exterior of shaft 1354 to transducer 1358. In one embodiment, the one or more wires may be disposed external to or embedded in an outer wall of shaft 1354. In use, distal end 1356 of shaft 1354 may be inserted through skin 1303 into a subcutaneous tissue plane 1305 to perform coagulation of the tissue plane 1305. While distal end 1356 is disposed in plane 1305, probe 1358 gathers acoustical data associated with tissue plane 1305 and provides the acoustical data to module 1362 to determine the mechanical properties of the tissue in and proximate to tissue plane 1305, such as the skin elasticity.

In either of the embodiments shown in FIGS. 13A, 13B, the acoustical data gathered by transducers 1300, 1358 may be provided to a generator, such as, generators 50, 1330, 1360 to perform method 150 described above. It is to be appreciated that in the embodiments of FIGS. 13A-B the effectiveness of an elasticity altering procedure may be determined by comparing the relative acoustical data measurements before treatment and measurements taken after subsequent treatments, for example, subsequent passes of a plasma device.

In another embodiment, a plasma applicator may be configured to test elasticity based on the mechanical strain imposed on the shaft of the applicator. For example, referring to FIG. 13C, a plasma applicator 1380 is shown in accordance with an embodiment of the present disclosure. Applicator 1380 is coupled to an ESU 1390 configured to provide electrosurgical energy to applicator 1380. Applicator 1380 includes a housing 1382 and a shaft having a distal end 1386, where plasma is applied to a tissue plane of interest 1305 via distal end 1386 when an inert gas is supplied to an energized electrode disposed within the shaft. Applicator 1380 further includes a strain sensor 1388 configured to sense strain (i.e., displacement) imposed on shaft 1384. In one embodiment, the strain sensor 1388 is disposed in housing 1382 and coupled to the shaft where the proximal end of the shaft has a pivot point in the housing 1382. When shaft 1384 is disposed through a tissue surface 1303 into subcutaneous tissue, an operator may rotate or tilt housing 1382 and shaft 1384 with respect to tissue surface 1303 and tissue 1305 to cause a mechanical strain to be imposed on shaft 1384, the strain imposed on shaft 1384 is detected by sensor 1388 and communicated to a processor in generator 1390. This process may be performed before and after a procedure (e.g., liposuction and/or coagulation) is performed on tissue 1305. Based on the amount of strain sensed before and after the procedure, the processor in generator 1390 is configured to determine the skin elasticity of tissue surface 1303 and/or the effectiveness of the elasticity altering procedure. It is to be appreciated that strain sensor 1388 measures displacement which has an inverse relationship with tissue tightness. For example, tighter tissue would have a lower strain and more laxity tissue would have a higher strain. In this way, applicator 1380 and generator 1390 may be used to perform the method 150 described above. For example, a baseline measurement may be taken with applicator 1380. A tissue elasticity altering procedure may then be performed, followed by a subsequent measurement with applicator 1380. If the measured strain is lower after the elasticity procedure, then the procedure was successful in reduce laxity of the tissue.

It is to be appreciated that, in one embodiment, applicator 1380 may further include a tilt sensor configured to determine a tilt angle of shaft 1384 with respect to a reference axis. In this way, when shaft 1384 is titled by user the tilt angle may be used with the strain sensed by sensor 1388 to normalize and compare strain measurements at different tilt angles and determine skin elasticity.

Referring to FIG. 13D, in another embodiment, applicator 1380 may further include probe/transducer 1358, e.g., a load cell, and analysis and display module 1362 for determining skin elasticity of the tissue plane 1305 in the manner described above. In this embodiment, applicator 1380 may measure both force from the load cell 1358 and the corresponding displacement using the strain sensor 1388 in the housing 1382. For example, the distal end 1386 of shaft 1384 may be employed to tent-up tissue where the probe/load cell 1358 may measure the force or stress at the distal end 1386. At the same time, the strain sensor 1388 may measure the strain on the shaft 1384 to determine the displacement of tissue. The force/stress and/or the strain/displacement may then be transmitted to the module 1362 and/or the generator 50 to determine the modulus of elasticity. Measurements can be taken before and after a procedure to perform the method 150 above.

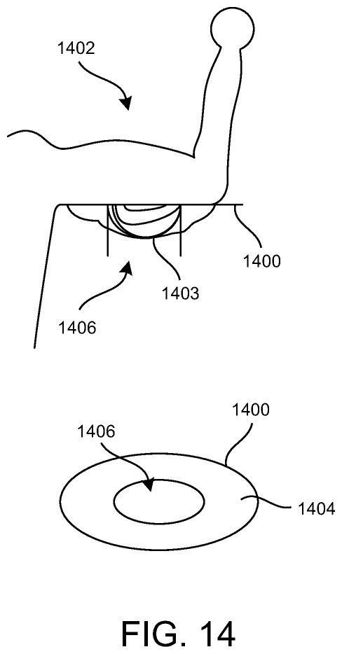

In another embodiment, skin elasticity may be determined by measuring the effect of gravity on a portion of patient tissue. For example, referring to FIG. 14, a device 1400 including surface 1404 (e.g., a planar surface) and an aperture 1406 (e.g., a circle or ellipse) having predetermined dimensions is shown. A portion of patient anatomy 1402 (e.g., the upper portion of a patient's arm) is placed on surface 1404, such that gravity pulls tissue of interest 1406 disposed within the perimeter of aperture 1406 through aperture 1406. Change in skin elasticity may be determined by measuring the distance the tissue of interest 1406 falls through the aperture 1406 (e.g., the distance below the surface 1404 that the tissue of interest is disposed) before and after a skin treatment (e.g., liposuction and/or plasma coagulation). The skin elasticity may then be provided to generator 50 to perform method 150 described above.

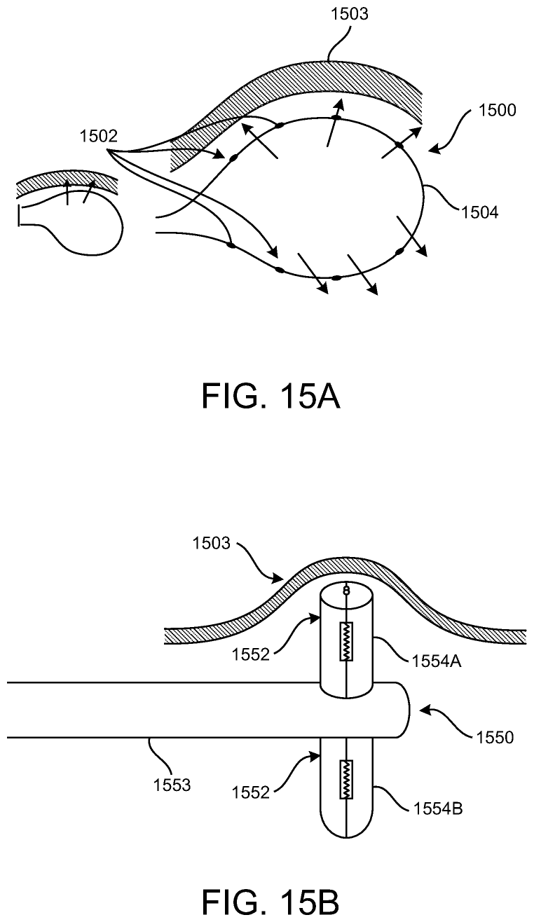

Referring to FIG. 15A, in another embodiment, the skin elasticity measurement device may be configured as an expanding device 1500 (e.g., a balloon or at least one inflatable element) having a plurality of pressure sensors 1502 disposed on an outer surface 1504 of the device 1500. Device 1500 may be made of an elastic material and coupled to a gas compressor for receiving and expelling gas to selectively inflate and deflate device 1500 to a desired size. When device 1500 is disposed in a subcutaneous tissue plane of interest below tissue surface 1503 and inflated, pressure sensors 1502 are configured to measure the pressure applied by the outer surface 1504 to displace the tissue in the tissue plane. The measurements may be taken before and after a procedure (e.g., liposuction and/or coagulation) performed in the tissue plane of interest. The measurements may be provided (e.g., via hardwired or wired connection) to generator 50 to determine the skin elasticity of skin 1503 and to perform method 150 described above. For example, in one embodiment, the device 1500 is introduced into the tissue plane of interest below surface 1503 and the pressure is measured to obtain a baseline measurement. The device 1500 is removed and a tissue altering procedure is performed. The device 1500 is then introduced again to get a post-treatment measurement. If the post-treatment measurement shows that the pressure is greater than the pressure measurement of the baseline, tissue firmness has increased.

It is to be appreciated that device 1500 may be modified in other embodiments such that that instead of the entirety of the device being inflatable, device 1500 includes distinct inflatable elements. For example, referring to FIG. 15B, a skin elasticity device 1500 is shown in accordance with another embodiment of the present disclosure. Device 1500 includes a shaft 1553 and a plurality of inflatable elements 1554A, 1554B, where each element 1554 includes a corresponding pressure sensor 1552 (e.g., included internally to each element 1554) configured to sense the pressure applied to elements 1553). Shaft 1553 is coupled to a gas compressor, where the compressor is used for inflating and deflating elements 1554 to a desired size. In one embodiment, elements 1554 extend perpendicularly away from shaft 1550 in opposite directions. In use, a portion of device 1550 is disposed in a subcutaneous tissue plane of interest and elements 1554 are inflated to a desired size. Sensors 1552 measure the pressure applied to elements 1554 by the surrounding tissue in the tissue plane. The measurements may be taken before and after a procedure (e.g., liposuction and/or coagulation) performed in the tissue plane of interest. The measurements are then provided (e.g., via hardwired or wired connection) to generator 50 to determine the skin elasticity of skin 1503 and to perform method 150 described above.

In another embodiment of the present disclosure, skin elasticity may be determined by monitoring the changes in impedance in patient tissue (e.g., on the tissue surface and/or in a tissue plane of interest) before and after a skin procedure or before and after skin has been displaced a known distance. The change in impedance may be used to determine the change in skin elasticity. It is to be appreciated that the change in impedance may be determined using impedance sensors contacting the tissue surface and/or tissue plane, which are communicatively coupled to generator 50. In one embodiment, the impedance sensors may be integrated in applicator 10 (e.g., disposed in distal end 16 of shaft 14). Alternatively, the change in impedance may be determined by generator 50 by sensing changes in electrosurgical energy provided to applicator 10.

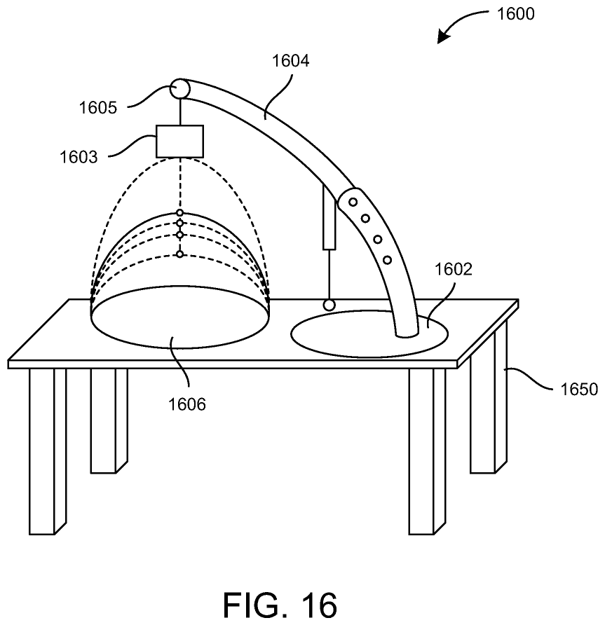

In another embodiment, the present disclosure provides a device, which may be placed on a surface of a structure, such as a table, and used to determine skin elasticity. For example, referring to FIG. 16, a device 1600 is shown in accordance with the present disclosure. Device 1600 includes a base 1602 and a rod 1604, which is coupled to and extends away from base 1602. Base 1602 may be placed on a surface 1650 of a structure (e.g., a table), such that a tip 1605 of rod 1604 extends a predetermined height above surface 1650. Tip 1605 is then attached to a pressure or force gauge 1603 and the force gauge 1603 is attached to a patient tissue surface 1606 (e.g., via an interface, such as an adhesive pad or suture loops, as described above). The pressure or force readings measured by the gauge 1603 before and after a treatment (e.g., liposuction and/or coagulation) of a tissue plane of interest may be used to determine the skin elasticity of surface 1606. The skin elasticity may be provided to generator 50 and used in method 150.

It is to be appreciated that in the embodiments above the make use of a force or pressure gauge, the force or pressure gauge may be any suitable force gauge, including, but not limited to, a strain resistor (e.g., measuring the change in voltage with strain) and a piezoelectrical device (e.g., a device measuring the change in conductance of a piezoelectrical material when the piezoelectrical material undergoes a strain). It is further to be appreciated that an exemplary force gauge may include at least one means for providing a measurement to a user and/or another device such as a generator, for example, the force gauge may include a display for displaying a measurement, a hardwire output connection for coupling the force gauge to another device, a wireless transceiver for wirelessly transmitting a measurement to another device, etc.

It is to be appreciated that any of the skin elasticity devices of the present disclosure may be used to provide skin elasticity measurements or other relevant information (e.g., tissue displacements, forces, etc.) to generator 50. Generator 50 may be coupled to a display and may output for display the current skin elasticity of a skin surface, a stress-strain curve, or any other relevant data. In one embodiment, if a processor in generator 50 determines that the slope of a stress-strain curve is constant (e.g., the skin elasticity is no longer changing), generator 50 outputs an alert or notification indicating that the skin surface and/or tissue pane of interest should no longer be displaced to prevent damage or permanent deformation of the patient tissue. In other embodiment, the generator 50 may output an alert or indication that a level of skin tightness has been achieved, for example, a skin firmness measurements is greater than a baseline measurement, a skin firmness measurement is greater than a predetermined skin firmness value, etc.

It is to be appreciated that the skin elasticity measurement devices of the present disclosure measure skin elasticity while taking into account the properties of tissue from the skin surface or dermis down to the muscle of the patient (i.e., through the entire FSN and not just the skin surface). To achieve this, for the devices that use the displacement of tissue surface to measure skin elasticity (e.g., devices 200, 300, 400, 500, 600, 900, 1200, 1400, 1600, 1700), a surface area above a predetermined size is selected for displacement. The predetermined size is established through experiment and observation. The remaining devices (e.g., 700, 800, 1000, 1100, 1500) are inserted into the tissue plane of interest and thus take into account the FSN.

It is to be appreciated that the terms “tissue plane”, “tissue plane of interest”, “subdermal tissue plane”, “subcutaneous tissue plane”, as used herein includes all layers or planes of patient tissue below the tissue surface or dermis of the patient and until the muscle.

It is to be appreciated that the various features shown and described are interchangeable, that is a feature shown in one embodiment may be incorporated into another embodiment.

While the disclosure has been shown and described with reference to certain preferred embodiments thereof, it will be understood by those skilled in the art that various changes in form and detail may be made therein without departing from the spirit and scope of the disclosure as defined by the appended claims.

Furthermore, although the foregoing text sets forth a detailed description of numerous embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

What is claimed is:

1. A method comprising:
performing an elasticity altering procedure on a subcutaneous tissue plane;
measuring elasticity of the altered tissue;
determining if the elasticity measurement is within a threshold value; and
iteratively performing an elasticity altering procedure on the subcutaneous tissue plane until the elasticity measurement is within the threshold.

2. The method of claim 1, further comprising determining the threshold value by measuring elasticity of the portion of the tissue before performing the elasticity altering procedure.

3. The method of claim 1, wherein the elasticity altering procedure is a liposuction procedure.

4. The method of claim 1, wherein the elasticity altering procedure involves applying energy to heat the tissue plane.

5. The method of claim 4, wherein the applying energy to heat the tissue plane includes applying a plasma beam to the tissue plane.

6. The method of claim 4, wherein the tissue plane is heated to a temperature of at least 85 degrees C. for about 0.04 seconds to about 0.08 seconds.

7. The method of claim 5, wherein a power output of the plasma beam is about 24 Watts to about 32 Watts.

8. The method of claim 1, wherein the measuring elasticity of the portion of the tissue plane includes:
measuring stress at a plurality of displacements of the tissue to obtain a plurality of data points; and
determining the slope of the data points.

9. The method of claim 8, wherein the determining if the elasticity measurement is within a threshold value includes comparing the determined slope to the threshold value.

10. The method of claim 1, wherein an initial elasticity altering procedure is a liposuction procedure and subsequent elasticity altering procedures include applying a plasma beam to the tissue plane.

11. A system comprising:
an applicator for applying a plasma beam to a tissue plane, the tissue plane beneath a portion of a surface of skin and receiving an elasticity altering procedure;
an elasticity measurement device for measuring elasticity of the portion of tissue; and
an electrosurgical generator unit for supplying electrosurgical energy to the applicator, determining if the elasticity measurement is within a threshold value and iteratively applying the plasma beam to the tissue plane until the elasticity measurement is within the threshold.

12. The system of claim 11, wherein the threshold value is the elasticity measurement of the portion of the tissue before the elasticity altering procedure is performed.

13. The system of claim 11, wherein the elasticity altering procedure is a liposuction procedure.

14. The system of claim 11, wherein the tissue plane is heated to a temperature of at least 85 degrees C. for about 0.04 seconds to about 0.08 seconds.

15. The system of claim 14, wherein a power output of the plasma beam is about 24 Watts to about 32 Watts.

16. The system of claim 11, wherein the elasticity measurement device measures elasticity by measuring stress at a plurality of displacements of the tissue to obtain a plurality of data points and determining the slope of the data points.

17. The system of claim 16, wherein the electrosurgical generator unit determines if the elasticity measurement is within the threshold value by comparing the determined slope to the threshold value.

18. The system of claim 11, wherein the elasticity measurement device is a tensile testing device.

19. The system of claim 11, wherein the elasticity measurement device is a vacuum assisted device.

20. The system of claim 11, wherein the elasticity measurement device is a lateral shear measurement device.

21. The system of claim 11, wherein the elasticity measurement device is a lateral load measurement device.

22. The system of claim 11, wherein the elasticity measurement device measures back pressure generated by gas introduced percutaneously to the tissue plane.

23. The system of claim 11, wherein the elasticity measurement device measures changes in density of a contrast fluid introduced into the tissue plane.

24. The system of claim 11, wherein the elasticity measurement device is pressure sensitive forceps.

25. The system of claim 11, wherein the elasticity measurement device is ultrasound device that introduces sound waves into the tissue plane and measures returning sounds waves propagating through the tissue plane.

26. The system of claim 11, wherein the elasticity measurement device includes at least one inflatable element, the at least one inflatable element includes a pressure sensor.
